SEQUENCE LISTING (16 sequences)

SEQ ID NO: 1 - DNA, 2802 bp, Staphylococcus aureus
atgaatatgaagaaaaaagaaaaacacgcaattcggaaaaaatcgattggcgtggcttcagtgcttgtaggtacgttaatcggttttggactactcagcagtaaagaagcagatgcaagtgaaaatagtgttacgcaatctgatagcgcaagtaacgaaagcaaaagtaatgattcaagtagcgttagtgctgcacctaaaacagacgacacaaacgtgagtgatactaaaacatcgtcaaacactaataatggcgaaacgagtgtggcgcaaaatccagcacaacaggaaacgacacaatcatcatcaacaaatgcaactacggaagaaacgccggtaactggtgaagctactactacgacaacgaatcaagctaatacaccggcaacaactcaatcaagcaatacaaatgcggaggaattagtgaatcaaacaagtaatgaaacgacttctaatgatactaatacagtatcatctgtaaattcacctcaaaattctacaaatgcggaaaatgtttcaacaacgcaagatacttcaactgaagcaacaccttcaaacaatgaatcagctccacagagtacagatgcaagtaataaagatgtagttaatcaagcggttaatacaagtgcgcctagaatgagagcatttagtttagcggcagtagctgcagatgcaccggcagctggcacagatattacgaatcagttgacgaatgtgacagttggtattgactctggtacgactgtgtatccgcaccaagcaggttatgtcaaactgaattatggtttttcagtgcctaattctgctgttaaaggtgacacattcaaaataactgtacctaaagaattaaacttaaatggtgtaacttcaactgctaaagtgccaccaattatggctggagatcaagtattggcaaatggtgtaatcgatagtgatggtaatgttatttatacatttacagactatgtaaatactaaagatgatgtaaaagcaactttgaccatgcccgcttatattgaccctgaaaatgttaaaaagacaggtaatgtgacattggctactggcataggtagtacaacagcaaacaaaacagtattagtagattatgaaaaatatggtaagttttataacttatctattaaaggtacaattgaccaaatcgataaaacaaataatacgtatcgtcagacaatttatgtcaatccaagtggagataacgttattgcgccggttttaacaggtaatttaaaaccaaatacggatagtaatgcattaatagatcagcaaaatacaagtattaaagtatataaagtagataatgcagctgatttatctgaaagttactttgtgaatccagaaaactttgaggatgtcactaatagtgtgaatattacattcccaaatccaaatcaatataaagtagagtttaatacgcctgatgatcaaattacaacaccgtatatagtagttgttaatggtcatattgatccgaatagcaaaggtgatttagctttacgttcaactttatatgggtataactcgaatataatttggcgctctatgtcatgggacaacgaagtagcatttaataacggatcaggttctggtgacggtatcgataaaccagttgttcctgaacaacctgatgagcctggtgaaattgaaccaattccagaggattcagattctgacccaggttcagattctggcagcgattctaattcagatagcggttcagattcgggtagtgattctacatcagatagtggttcagattcagcgagtgattcagattcagcaagtgattcagactcagcgagtgattcagattcagcaagcgattccgactcagcgagcgattccgactcagacaatgactcggattcagatagcgattctgactcagacagtgactcagattccgacagtgactcagattcagatagcgattctgactcagacagtgactcagattcagatagcgattcagattcagatagcgattcagattccgacagtgattccgactcagacagcgattctgactccgacagtgattccgactcagacagcgattcagattccgacagtgattccgactcagatagcgattccgactcagatagcgactcagattcagacagcgattcagattcagacagcgattcagattcagatagcgattcagattccgacagtgactcagattccgacagtgactcggattcagatagcgattcagattccgacagtgactcagattccgacagtgactcagactcagacagtgattcggattcagcgagtgattcggattcagatagtgattccgactccgacagtgactcggattcagatagcgactcagactcggatagcgactcggattcagatagcgattcggactcagatagcgattcagaatcagacagcgattcagaatcagacagcgattcagattcagacagcgactcagacagtgactcagattcagatagtgactcggattcagcgagtgattcagactcaggtagtgactccgattcatcaagtgattccgactcagaaagtgattcaaatagcgattccgagtcaggttctaacaataatgtagttccgcctaattcacctaaaaatggtactaatgcttctaataaaaatgaggctaaagatagtaaagaaccattaccagatacaggttctgaagatgaagcaaatacgtcactaatttggggattattagcatcaataggttcattactacttttcagaagaaaaaaagaaaataaagataagaaataa

SEQ ID NO: 2 - protein, 933 aa, Staphylococcus aureus
MNMKKKEKHAIRKKSIGVASVLVGTLIGFGLLSSKEADASENSVTQSDSASNESKSNDSSSVSAAPKTDDTNVSDTKTSSNTNNGETSVAQNPAQQETTQSSSTNATTEETPVTGEATTTTTNQANTPATTQSSNTNAEELVNQTSNETTSNDTNTVSSVNSPQNSTNAENVSTTQDTSTEATPSNNESAPQSTDASNKDVVNQAVNTSAPRMRAFSLAAVAADAPAAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAYIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPEDSDSDPGSDSGSDSNSDSGSDSGSDSTSDSGSDSASDSDSASDSDSASDSDSASDSDSASDSDSDNDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSASDSDSDSDSDSDSDSDSDSDSDSDSDSDSDSDSDSDSDSESDSDSESDSDSDSDSDSDSDSDSDSDSDSASDSDSGSDSDSSSDSDSESDSNSDSESGSNNNVVPPNSPKNGTNASNKNEAKDSKEPLPDTGSEDEANTSLIWGLLASIGSLLLFRRKKENKDKK

SEQ ID NO: 3 - DNA, 43 bp, Staphylococcus aureus
cgcggatccatagtaacaaaggattatagtgggaaatcacaag

SEQ ID NO: 4 - DNA, 39 bp, Staphylococcus aureus
tcccccgggttattttgttactctaggcccatatgtcgc

SEQ ID NO: 5 - DNA, 32 bp, Staphylococcus aureus
cgcggatccgtggtttctggggagaagaatcc

SEQ ID NO: 6 - DNA, 36 bp, Staphylococcus aureus
tcccccgggtttgcagccatgcattaattatttgcc

SEQ ID NO: 7 - DNA, 27 bp, Staphylococcus aureus
cgcggatccaaggtcaaatcgaccgtt

SEQ ID NO: 8 - DNA, 38 bp, Staphylococcus aureus
cggggtaccttatttcttatctttattttctttttttc

SEQ ID NO: 9 - protein, 121 aa, Artificial Sequence
  Synthetic peptide
QVQLKESGPGLVAPSQSLSITCAISGFSLSRYSVHWVRQPPGKGLEWLGMIWGGGNTDYNSALKSRLSISKDNSKSQVFLKMNSLQTDDTAMYYCARKGEFYYGYDGFVYWGQGTLVTVSA

SEQ ID NO: 10 - protein, 118 aa, Artificial Sequence
  Synthetic peptide
QVHLKESGPGLVAPSQSLSITCTVSGFSLSRYNIHWVRQPPGKGLEWLGMIWGGENTDYNSALKSRLSISKDNSKSQVFLKMNSLQTDDTAMYYCASAYYGNSWFAYWGQGTLVTVSA

SEQ ID NO: 11 - protein, 115 aa, Artificial Sequence
  Synthetic peptide
QVQLKESGPGLVAPSQSLSITCTVSGFSLSRYSVHWVRQPPGKGLEWLGMIWGGGSTDYNSALKSRLNISKDNSKSQVFLKMNSLQTDDTAMYYCARRLWYFDVWGAGTTVTVSS

SEQ ID NO: 12 - protein, 118 aa, Artificial Sequence
  Synthetic peptide
QVQLKESGPGLVAPSQSLSITCTVSGFSLSRYSVHWVRQPPGKGLEWLGMIWGGGNTDYNSALKSRLSITKDNSKSQVFLKMNSLQTDDTAMYYCATAYYGNSWFAYWGQGTLVTVSA

SEQ ID NO: 13 - protein, 112 aa, Artificial Sequence
  Synthetic peptide
NIMMTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSYTFGGGTKLEIK

SEQ ID NO: 14 - protein, 112 aa, Artificial Sequence
  Synthetic peptide
NIMMTQSPSSLAVSAGEKVTMSCKSSQSVLYSSNQKNYLAWYQQKPGQSPKLLIYWASTRESGVPDRFTGSGSGTDFTLTINSVQAEDLAVYYCHQYLSSHTFGGGTKLEIK

SEQ ID NO: 15 - protein, 112 aa, Artificial Sequence
  Synthetic peptide
NIMMTQSPSSLAVSAGEKVTMSCKSSQSVLYSSNQKNYLAWYQQKPGQSPKLLIYWASTRESGVPDRFTGSGSGTDFTLTISSVQAEDLAVYCCHQYLSSYTFGGGTELEIK

SEQ ID NO: 16 - protein, 112 aa, Artificial Sequence
  Synthetic peptide
NIMMTQSPSSLAVSAGEKVTMSCRSSQSVLYSSNQKNYLAWYQQKPGQSPTLLIYWASTRESGVPDRFTGSGSGTDFTLTISSVQAEDLAVYYCHQYLSSYTFGGGTKLEIK

(19) United States
(12) Patent Application Publication
(10) Pub. No.: US 2014/0271649 A1
(43) Pub. Date: Sep. 18, 2014

(54) METHODS AND COMPOSITIONS FOR INHIBITING STAPHYLOCOCCUS AGGLUTINATION IN BLOOD

(71) Applicants: Olaf Schneewind, Chicago, IL (US); Dominique Missiakas, Chicago, IL (US); Molly Mcadow, Chicago, IL (US); Hwan Keun Kim, Chicago, IL (US)

(72) Inventors: Olaf Schneewind, Chicago, IL (US); Dominique Missiakas, Chicago, IL (US); Molly Mcadow, Chicago, IL (US); Hwan Keun Kim, Chicago, IL (US)

(73) Assignee: The University of Chicago, Chicago, IL (US)

(21) Appl. No.: 14/233,109

(22) PCT Filed: Jul. 13, 2012
(86) PCT No.: PCT/US2012/046739

§ 371 Date: May 27, 2014

Related U.S. Application Data
(60) Provisional application No. 61/508,430, filed on Jul. 15, 2011.
(60) Provisional application No. 61/530,869, filed on Sep. 2, 2011.

(51) Int. Cl. A61K 39/40; A61K 31/4439; A61K 31/4545

(52) U.S. Cl. CPC A61K 39/40; A61K 31/4545; A61K 31/4439
USPC 424/136.1; 424/133.1; 424/165.1

(57) ABSTRACT

Certain embodiments are directed to methods of inhibiting Staphylococcal infection comprising administering effective amounts of a ClfA inhibitor and a thrombin inhibitor to a patient.

This application claims the benefit of U.S. Provisional Patent Application Nos. 61/508,430, filed on Jul. 15, 2011, and 61/530,869, filed on Sep. 2, 2011, each of which is incorporated herein by reference in its entirety.

This invention was made with government support under AI52474, AI92711, and AI52767 awarded by the National Institute of Allergy and Infectious Diseases and under AI057153 awarded by the National Institutes of Health. The government has certain rights in the invention.

### BACKGROUND OF THE INVENTION

A. Field of the Invention

Embodiments of this invention are directed generally to microbiology and medicine. In certain aspects the invention is directed to prevention and/or treatment of Staphylococcus infection.

B. Background

The Gram-positive bacterium Staphylococcus aureus is the causative agent of human skin and soft tissue infections, invasive disease and bacteremia (Lowy, 1998). Staphylococcal bacteremia leads to endocarditis and sepsis, diseases that, even under antibiotic therapy, are associated with very high mortality (Klevens et al., 2007). Community- and hospital-acquired infections are frequently caused by antibiotic (methicillin)-resistant S. aureus (MRSA), resulting in poor disease outcomes following the failure of antibiotic therapy (Fowler et al., 2005).

S. aureus is a unique disease pathogen owing to its multiple interactions with fibrinogen (Cheng et al., 2011), a highly abundant host protein responsible for the formation of fibrin clots following cleavage by thrombin (Doolittle, 2003). S. aureus secretes coagulases, polypeptides that activate prothrombin to cleave the Aα and Bβ chains of fibrinogen, thereby generating fibrin clots (Friedrich et al., 2003). This reaction, analyzed as the coagulation of calcium-chelated plasma following incubation with bacteria (Cheng et al., 2010), is used in clinical laboratories to distinguish S. aureus isolates from non-pathogenic microbes (coagulase test) (Loeb, 1903). Another diagnostic tool, the slide agglutination test, monitors the agglutination of S. aureus immersed in calcium-chelated plasma (Kolle and Otto, 1902). The biochemical attributes and physiological relevance of staphylococcal agglutination are not yet known. Moreover, a preventive or treatment strategy that can reduce the burden and improve the outcomes of S. aureus sepsis is urgently needed.

### SUMMARY OF THE INVENTION

Staphylococcus aureus remains a leading cause of infectious disease morbidity and mortality. Accordingly, compositions and methods are provided to prevent and/or treat Staphylococcus bacteria infection in a subject.

In some embodiments there are methods for treating or preventing a Staphylococcus bacteria infection in a subject comprising administering to the subject effective amounts a ClfA inhibitor and a thrombin inhibitor so as to attempt to treat or prevent infection by the Staphylococcus bacteria. In some embodiments, methods involving both a ClfA inhibitor and a thrombin inhibitor that are administered within 24 hours of each other are referred to as a “combination inhibitor therapy.”

In additional embodiments, there are methods for treating or preventing infection by a Staphylococcus aureus bacteria comprising administering to the subject effective amounts a ClfA inhibitor and a thrombin inhibitor.

In further embodiments, there are methods for inhibiting Staphylococcus agglutination in a subject comprising administering to the subject effective amounts a ClfA inhibitor and a thrombin inhibitor.

In particular embodiments, there are methods for treating or preventing a Staphylococcus aureus infection in a human patient comprising administered to the patient effective amounts of a ClfA inhibitor and a thrombin inhibitor, wherein the ClfA inhibitor is a chimeric or humanized antibody or an antibody fragment.

In other embodiments, there are compositions comprising a ClfA inhibitor and a thrombin inhibitor.

In certain embodiments, the subject is any organism that Staphylococcus infects and that leads to an illness, a condition, a disease, and has a detrimental health consequence, such as necrosis, cellular damage, tissue damage, or even death. In some embodiments, the subject is a mammal. In further embodiments, the mammal is a cow, sheep, pig, dog, cat, goat, mouse, rat, rabbit, horse, or monkey. In specific embodiments, the subject is a human.

In certain embodiments the subject has been exposed to Staphylococcus bacteria or is at risk for exposure to Staphylococcus bacteria. In other embodiments, the subject is suspected of having been infected with or has been determined to be infected with a Staphylococcus bacteria. In certain embodiments, the bacteria is Staphylococcus aureus. In a further aspect the Staphylococcus aureus infection is a drug resistant Staphylococcus aureus infection. In even further embodiments, the Staphylococcus bacteria is methicillin-resistant, including Staphylococcus aureus methycillin-resistant (MRSA) bacteria.

In certain aspects the patient is determined to have a Staphylococcal infection. The methods can further comprise identifying the patient as having a Staphylococcal infection. In a further aspect the method can further comprise selecting the patient after the patient is diagnosed with a Staphylococcal infection. The method can also further comprise the step of testing the patient for a Staphylococcal infection or having the patient tested for Staphylococcal infection. The method can include obtaining from the patient a biological sample for testing whether the patient has a Staphylococcal infection. In a further aspect the patient has a Staphylococcus infection, which includes but is not limited to pneumonia, sepsis, corneal infection, skin infection, infection of the central nervous system, or toxic shock syndrome.

Certain embodiments are directed to methods where the patient is immune deficient, is immunocompromised, is hospitalized, is undergoing an invasive medical procedure, is infected with influenza virus and/or is on a respirator. In specific embodiments, the invasive medical procedure involves surgery. In some methods, a patient will undergo surgery within 1, 2, 3, 4, 5, 6, or 7 days of administering or being administered a ClfA inhibitor and/or a thrombin inhibitor. In specific aspects, a patient will be placed on a respirator within 1, 2, 3, 4, 5, 6, or 7 days of administering or being administered a ClfA inhibitor and/or a thrombin inhibitor.

In certain aspects the methods can further comprise the step of monitoring the patient for a Staphylococcal infection within a week of administering an inhibitor.

In certain embodiments, the subject is administered the ClfA inhibitor and/or a thrombin inhibitor upon being determined to have a Staphylococcus bacterial infection. In certain embodiments, the subject is administered the ClfA inhibitor and/or a thrombin inhibitor within 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, and/or 1, 2, 3, 4, 5, 6, or 7 days of being determined to have a Staphylococcus bacterial infection. In some embodiments, a subject is administered the ClfA inhibitor and/or a thrombin inhibitor within 12 hours, 24 hours, or 48 hours of being determined to be infected with Staphylococcus bacteria.

The term “effective amounts” means that the amounts of each of the ClfA inhibitor and a thrombin inhibitor that have been effective for treating or preventing infection by a Staphylococcus bacteria when both a ClfA inhibitor and at least one thrombin inhibitor or both a ClfA inhibitor and a combination of thrombin inhibitors are administered to a subject. In particular embodiments, the subject is given at least or at most 1, 2, 3, 4, 5, 6, 7, 8, 9, 10, 11, 12, 13, 14, 15, 16, 17, 18, 19, 20 or more doses of the ClfA inhibitor. In certain embodiments, the subject is given at least or at most 1, 2, 3, 4, 5, 6, 7, 8, 9, 10, 11, 12, 13, 14, 15, 16, 17, 18, 19, 20 or more doses of the thrombin inhibitor(s).

A thrombin inhibitor refers to a compound or substance that inhibits thrombin activity, such as its serine protease activity. In certain embodiments, the thrombin inhibitor is a direct thrombin inhibitor, which means it binds directly to thrombin. In further embodiments, the thrombin inhibitor is a univalent direct thrombin inhibitor, which means it only binds to the active site of thrombin. In some embodiments, the univalent direct thrombin inhibitor is argatroban, dabigatran, melagatran (or its prodrug, ximelagatran), inogatran, efegatran, hirudin, bivalirudin, odiparcil or efegatran-sulfate. In specific embodiments, the thrombin inhibitor is dabigatran (also known as dabigatran extexilate). In other embodiments, the thrombin inhibitor is argatroban, while in even further embodiments, the thrombin inhibitor is melagatran (or its prodrug, ximelagatran). In specific embodiments, it is melagatran.

In further embodiments, more than one different thrombin inhibitor is administered to the subject. In specific embodiments, at least two thrombin inhibitors are administered to the subject. In some embodiments, two different thrombin inhibitors are administered. In other embodiments, at least three different thrombin inhibitors are administered to the subject. When more than one thrombin inhibitor is employed, the thrombin inhibitor may be any of the inhibitors discussed herein, but is not limited to these inhibitors. It is contemplated that when more than one different thrombin inhibitor is administered, they may be administered to the subject separately, which means they may be administered at different times and/or in different solutions. In certain embodiments, two or more thrombin inhibitors may be administered together to the subject. It is also contemplated that when multiple thrombin inhibitors are administered that some may be administered together but another or others may be administered separately. It is also contemplated that one or more dosings may involve the administration of two or more thrombin inhibitors together but that other dosings may not.

It is contemplated that a thrombin inhibitor may be administered at one time and then the same or a different thrombin inhibitor is administered within 30 minutes to the subject. In other embodiments, it is contemplated that a thrombin inhibitor may be administered at one time and then the same or a different thrombin inhibitor is administered 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, and/or 1, 2, 3, 4, 5, 6, or 7 days within each other, and any duration of time derivable therein. It is also contemplated that one thrombin inhibitor may be administered to a subject one or more times and that subsequently at least one different thrombin inhibitor is administered one or more times. In certain embodiments, different thrombin inhibitors may be administered one or more times to the subject.

A ClfA inhibitor is a compound or agent that inhibits or reduces ClfA activity, such as binding to fibrinogen or fibrin or more generally involvement or promotion of Staphylococcus agglutination. In certain embodiments, the ClfA inhibitor is a small molecule, protein, or nucleic acid. In specific embodiments, the ClfA inhibitor is a protein, which refers to a polypeptide or a peptide. In certain embodiments, the ClfA inhibitor is an isolated antibody or antibody fragment that specifically binds ClfA. In some embodiments, the antibody is chimeric, humanized, scFv, or bi-specific. In particular embodiments, the antibody is chimeric or humanized. It is specifically contemplated that any embodiment discussed in the context of a “ClfA inhibitor” may be implemented specifically with a ClfA inhibitor that is an antibody or an antibody fragment. In certain methods or compositions, the ClfA inhibitor blocks the interaction of ClfA with fibrin. In additional methods and compositions, the ClfA inhibitor blocks the interaction of ClfA with fibrinogen. In some methods and compositions, the ClfA inhibitor blocks the interaction of ClfA with both fibrinogen and fibrin.

Thus, in some aspects, a ClfA inhibitor is an antibody or a fragment thereof that binds to ClfA and reduces ClfA binding to fibrinogen or fibrin or reduces ClfA-mediated agglutination. For example, the ClfA-binding antibody can be a monoclonal antibody or a recombinant antibody that comprises the heavy and light chain variable domain CDRs from a ClfA-binding monoclonal antibody. Examples of ClfA-binding antibodies that can be used in accordance with the embodiments include, without limitation, the antibodies (or antibody CDR domains) provided detailed in Hall et al., 2003, U.S. Pat. Nos. 6,979,446; 6,692,739; 6,692,739; 7,045,131; 7,364,738 and U.S. Pat. Publn. No. US20060222651, each of which is incorporated herein by reference in its entirety.

Aspects of the embodiments are directed to monoclonal antibody polypeptides, polypeptides having one or more segments thereof, and polynucleotides encoding the same. Thus, in certain aspects, a polypeptide can comprise all or part of the heavy chain variable region and/or the light chain variable region of ClfA specific antibodies (e.g., antibodies that bind to ClfA and inhibit Clfa-mediated agglutination). In a further aspect, a polypeptide can comprise an amino acid sequence that corresponds to a first, second, and/or third complementary determining regions (CDRs) from the light variable chain and/or heavy variable chain of an antibody, e.g., a ClfA-binding antibody.

In certain aspects, a polypeptide comprises all or part of an amino acid sequence corresponding to the MAb 12-9, 13-2, 35-006 or 35-220 variable (VDJ) heavy chain amino acid sequence 12-9 (QVQLKESGPGLVAPSQSLSITCAISGFSLSRYSVHWVRQPPGKGLEWLGMIWGGGN TDYNSALKSRLSISKDNSKSQVFLKMNSLQTDDTAMYYCARKGEFYYGYDGFVYW GQGTLVTVSA) (SEQ ID NO:9); 13-2 (QVHLKESGPGLVAPSQSLSITCTVSGFSLSRYNIHWVRQPPGKGLEWLGMIWGGEN TDYNSALKSRLSISKDNSKSQVFLKMNSLQTDDTAMYYCASAYYGNSWFAYWGQG TLVTVSA) (SEQ ID NO:10); 35-006 (QVQLKESGPGLVAPSQSLSITCTVSGFSLSRYSVHWVRQPPGKGLEWLGMIWGGGS TDYNSALKSRLNISKDNSKSQVFLKMNSLQTDDTAMYYCARRLWYFDVWGAGTTV TVSS) (SEQ ID NO:11); 35-220 (QVQLKESGPGLVAPSQSLSITCTVSGFSLSRYSVHWVRQPPGKGLEWLGMIWGGGN TDYNSALKSRLSITKDNSKSQVFLKMNSLQTDDTAMYYCATAYYGNSWFAYWGQG TLVTVSA) (SEQ ID NO:12). CDRs are indicated in bold underline. CDRs are regions within antibodies where the antibody complements an antigen's shape, and thus, determine the protein's affinity and specificity for specific antigens. From amino to carboxy terminus the CDRs are CDR1, CDR2, and CDR3. In certain aspects, a polypeptide can comprise 1, 2, and/or 3 CDRs from the variable heavy chain of MAb 12-9, 13-2, 35-006, 35-220 or a combination thereof.

In certain aspects, a polypeptide comprises all or part of an amino acid sequence corresponding to the MAb 12-9, 13-2, 35-006 or 35-220 variable (VJ) light chain amino acid sequence 12-9 (NIMMTQSPSSLAVSAGEKVTMSCKSSQSVLYSSNQKNYLAWYQQKPGQSPK LLIYWASTRESGVPDRFTGSGSGTDFTLTISSVQAEDLAVYYCHQYLSSYTFGGGTKL EIK) (SEQ ID NO:13); 13-2 (NIMMTQSPSSLAVSAGEKVTMSCKSSQSVLYSSNQKNYLAWYQQKPGQSPK LLIYWASTRESGVPDRFTGSGSGTDFTLTINSVQAEDLAVYYCHQYLSSHTFGGGTK LEIK) (SEQ ID NO:14); 33-006 (NIMMTQSPSSLAVSAGEKVTMSCKSSQSVLYSSNQKNYLAWYQQKPGQSPKLLIY WASTRESGVPDRFTGSGSGTDFTLTISSVQAEDLAVYCCHQYLSSYTFGGGTELEIK) (SEQ ID NO:15); 35-220 (NIMMTQSPSSLAVSAGEKVTMSCRSSQSVLYSSNQKNYLAWYQQKPGQSPTLLIYW ASTRESGVPDRFTGSGSGTDFTLTISSVQAEDLAVYYCHQYLSSYTFGGGTKLEIK) (SEQ ID NO:16). CDRs are indicated in bold underline. From amino to carboxy terminus the CDRs are CDR1, CDR2, and CDR3. In certain aspects, a polypeptide can comprise 1, 2, and/or 3 CDRs from the variable light chain of MAb 12-9, 13-2, 35-006, 35-220 or a combination thereof.

In some embodiments, the ClfA inhibitor and at least one thrombin inhibitor are administered to the subject in a composition together. In certain embodiments, a composition includes the ClfA inhibitor and thrombin inhibitors, and the composition is administered to the subject. In other embodiments, the ClfA inhibitor is administered to the subject prior to the administration of a thrombin inhibitor. In further embodiments, a thrombin inhibitor is administered to the subject prior to the administration of the ClfA inhibitor.

Certain embodiments further comprise administering an additional anti-microbial agent or treatment. In certain aspects the additional anti-microbial agent or treatment is an antibiotic agent, an anti-infective agent, a passive vaccine, or an active vaccine. In specific embodiments, methods include administering an antibiotic to the subject. The additional antimicrobial agent or treatment may have been given prior to administration of either a ClfA inhibitor or a thrombin inhibitor. In other embodiments, the antimicrobial is given after or at the same time as a ClfA inhibitor and/or a thrombin inhibitor. In specific embodiments, an antibiotic is given before, after and/or at the same time as a ClfA inhibitor and/or a thrombin inhibitor.

It is contemplated that a subject may be administered a ClfA inhibitor, a thrombin inhibitor, or an antibiotic orally, parenterally, subcutaneously, intramuscularly, or intravenously (or a combination thereof). In some embodiments, one or more of the compounds may be infused into a subject and/or administered as a bolus to the subject. In a further aspect, an inhibitor and/or antibiotic is administered orally, topically, intravascularly, intrathecally, intratracheally, by inhalation, or by instillation. The inhibitor or antibiotic can be administered directly to various organs or tissues including, but not limited to the subject's skin, respiratory tract (including the lungs), kidneys, central nervous system, reproductive organs, vagina, or eyes.

In certain embodiments, a subject is administered at least or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 milligrams (mgs) or micrograms (mcg) or μg/kg or micrograms/kg/minute, or any range derivable therein. In specific embodiments, a subject is administered about 75 to 150 mg of a thrombin inhibitor, such as dabigatran or argatroban, that is administered intravenously twice a day.

In other embodiments, a thrombin inhibitor is administered 2 mcg/kg/min by infusion. In other embodiments, such as for percutaneous coronary intervention (PCI) an infusion is started and a bolus is administered via a large bore intravenous (IV) line over about 3 to 5 minutes. In certain embodiments, the infusion is started at 350 mcg/kg and the bolus is 350 mcg/kg. In other particular embodiments, methods involve taking about 24-36 mg of a thrombin inhibitor, such as melagatran/ximelagatran orally twice daily.

For argatroban, initial dosing may be 0.5, 1.0, 1.5, 2.0, or 2.5 mg/kg/min, which may be continued until steady state aPTT levels are 1.5 to 3.0 patient's baseline values.

For dabigatran etexilate mesylate, in some embodiments a 150 mg capsule is administered or taken twice daily.

It is contemplated that methods may involve adjusting dosage after the initial dosing period. In some embodiments, a dosing adjustment may follow a test to determine the steady state level of the dosed drug. In certain embodiments, methods may involve monitoring coagulation in a patient before and/or after therapy with a thrombin inhibitor.

For ximelagatran, 24 mg tablets may be taken or administered to a patient twice daily.

Other embodiments concern compositions comprising both a ClfA inhibitor and a thrombin inhibitor. In certain embodiments, the ClfA inhibitor in the composition is an antibody or antibody fragment. In particular embodiments the ClfA inhibitor is a humanized or chimeric antibody. In some embodiments, the thrombin inhibitor is argatroban, dabigatran, melagatran (or its prodrug, ximelagatran), inogatran, or efegatran, or a salt or prodrug thereof. It is contemplated that a composition may contain more than one thrombin inhibitor.

The amounts of each may be about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 milligrams (mgs) or micrograms (mcg), or any range derivable therein. The composition may be have a form that is solid, liquid, gel, or semisolid. The composition may include other inactive or active ingredients discussed herein.

The volume of the composition may be about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 microliters, milliliters, or deciliters, or any range derivable therein.

Other embodiments of the invention are discussed throughout this application. Any embodiment discussed with respect to one aspect applies to other aspects as well and vice versa. The embodiments in the Example section are understood to be embodiments of the invention that are applicable to all aspects of the invention.

It is specifically contemplated that any embodiment discussed in the context of a ClfA inhibitor may be applied with a thrombin inhibitor or with a combination inhibitor therapy, and vice versa. It is specifically contemplated that any embodiment discussed in the context of a thrombin inhibitor may be applied with a ClfA inhibitor or with a combination inhibitor therapy, and vice versa.

The terms “inhibiting,” “reducing,” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.” It is also contemplated that anything listed using the term “or” may also be specifically excluded.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

It is specifically contemplated that any embodiment described as “comprising” certain components may also be implemented as “consistenting essentially of” those components, where “consisting essentially of” refers to the active ingredient relative to inactive or contaminating compounds that may be in a composition and/or method.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1C. Staphylococcus aureus agglutination in blood is a multi-factorial process and essential for the pathogenesis of sepsis in mice. (A) Agglutination in EDTA-plasma of Syto-9 stained S. aureus Newman wild-type (wt) or its isogenic mutants with insertional lesions in single or multiple genes: coa (coagulase), vwb (von Willebrand factor binding protein), clfA (clumping factor A), clfB and efb (extracellular fibrinogen binding protein). (B) Quantification of agglutination for staphylococcal mutants (A) expressed as the percent relative to wt (100%). Average and standard error of the means were calculated from sixteen fields of microscopic view and statistical significance was assessed in pairwise comparison between wt and mutant with the two-tailed Student's t-test: *P<0.01, **P<0.0001. (C) Complementation studies of staphylococcal agglutination using the slide agglutination test. S. aureus Newman variants coa/vwb and clfA were transformed with plasmids pcoa-vwb and pclfA, respectively. Statistical significance was analyzed by two-tailed Student's t-test; ***P<0.0001.

FIGS. 2A-2E. Staphylococcus aureus agglutination occurs during the pathogenesis of sepsis in mice. Quantification of heart lesions in BALB/c mice (n=10) 12 hours post-infection with S. aureus Newman. Three types of lesions were observed with either (A) staphylococcal agglutination (SA) without immune cell infiltrates (PMNs, polymorphonuclear leukocytes), (B) immune cell infiltrates without SAs (PMNs only) or (C) SA with surrounding granulocytes (SA+PMNs). Heart tissues were stained with hematoxylin-eosin and lesions enumerated (D). Error bars represent standard error of the mean of tissue samples. Data are representative of two independent experiments. (E) Immuno-histochemical analysis of heart tissues from BALB/c mice (n=10) 12 hours following intravenous challenge with S. aureus Newman. Samples were stained with antibodies directed against mouse fibrinogen (α-fibrinogen) or mouse prothrombin (α-prothrombin). Arrows point to staphylococcal agglutinations (black) or immune cell infiltrates (green); scale bars represent 1 μm.

FIGS. 3A-3C. Staphylococcal agglutination in heart tissues is required for the pathogenesis of sepsis. (A) Staphylococcal load, enumerated as colony forming units (CFU), in heart tissues of BALB/c mice (n=10) 12 hours after retro-orbital inoculation with 108 CFU of S. aureus Newman (wt) or its variant strains (clfA, coa/vwb and coa/vwb/clfA). Horizontal lines represent mean CFU. Statistical analysis was performed with the Mann-Whitney test: wt vs. clfA, P=0.0002; wt vs. coa/vwb, P=0.0002; wt vs. coa/vwb/clfA, P=0.0002; coa/vwb vs. coa/vwb/clfA, P=0.0007; clfA vs. coa/vwb/clfA, P=0.0002. Data are representative of two independent experiments. (B) Summary of histopathology findings in thin-sectioned and hematoxylin-eosin stained heart tissue from BALB/c mice (n=10) 12 hours after retro-orbital injection of S. aureus Newman wild-type (wt) or its clfA, coa/vwb as well as clfA/coa/vwb variants. Representative lesions in heart tissues included staphylococcal agglutination without PMNs (SA), with PMNs (SA+PMNs), and PMN accumulation without staphylococcal agglutination (PMNs−SA). Error bars represent standard error of the mean from 10 hearts. Statistical significance of lesions for each mutant compared to wt infection was determined by Student's t test: *P<0.05, **P<0.01, ***P<0.001. Data are representative of two independent experiments. (C) Survival of cohorts of BALB/c mice (n=20) following intravenous injection with S. aureus Newman (wt) or variants lacking coa, vwb or clfA. Data are representative of three independent experiments. Statistical significance was assessed with the logrank test: wt vs. coa/vwb (P<0.01), wt vs. clfA (P<0.001), and wt vs. coa/vwb/clfA (P<0.0001).

FIGS. 4A-4D. ClfA enables staphylococcal agglutination with fibrin cables in vitro and in vivo. (A) The association of purified recombinant ClfA with immobilized fibrinogen or fibrin was assessed by ELISA and analyzed as the percentage of maximal binding. Average and standard error of the means were calculated from three independent experiments. Curves represent nonlinear regression for one-site binding saturation performed with GraphPad Prism, Fbgn R2=0.9876; Fibrin R2=0.9876. (B) Scanning electron micrographs of S. aureus Newman (wt) and its isogenic mutants immersed in plasma. (C) Affinity-purified rabbit IgG specific for Coa (α-Coa), vwb (α-vWb), ClfA (α-ClfA) or the plague protective antigen V10 (α-V10) was analyzed for its ability to prevent staphylococcal agglutination. Statistical significance of antibody effects compared to a mock treated control was assessed with the Student's t test: *P<0.05. (D) BALB/c mice (n=10) were passively immunized by intraperitoneal injection with affinity-purified antibodies against V10 or ClfA and disease protection assessed by intravenous challenge with S. aureus Newman. Data represent one of three independent experiments. Statistical significance was assessed with the logrank test: P<0.01.

FIGS. 5A-5C. Neutralization of coagulases and ClfA prevents staphylococcal agglutination in heart tissues of septic mice. (A) Quantification of histopathology lesions in heart tissues of BALB/c mice (n=10) passively immunized with affinity-purified V10 control antibodies (which neutralize the plague protective antigen LcrV) or ClfA antibodies prior to lethal infection. Hearts were removed during necropsy 12 hours after retro-orbital inoculation of staphylococci. Tissues were thin-sectioned, stained with hematoxylin-eosin and histopathology lesions enumerated. Error bars represent standard error of the mean from cohorts of ten mice. Statistical analysis was performed by two-tailed Student's t-test comparing same lesion types between mock-immunized and vaccinated animals: *P<0.05, **P<0.01, ***P<0.001. (B) Quantification of three types of histopathology lesions in heart tissues from mice actively immunized with recombinant Coa, vWbp, or ClfA. Hearts were removed during necropsy 12 hours after retro-orbital inoculation of staphylococci into BALB/c mice (n=10). Tissues were thin-sectioned, stained with hematoxylin-eosin and histopathology lesions enumerated. Error bars represent standard error of the mean from cohorts of ten mice. Statistical analysis was performed by Student's two-tailed t-test comparing same lesion types between mock-immunized and vaccinated animals: *P<0.05, **P<0.01, ***P<0.001. Data are representative of two independent experiments. (C) Half maximal IgG antibody titer specific for Coa, vWb or ClfA antigens in serum following active vaccination of BALB/c mice (n=5). Blood samples were drawn at the time of challenge. Error bars represent standard deviation of serum IgG titers. The limit of detection is 100.

FIGS. 6A-6D. Direct thrombin inhibitors block a key step in staphylococcal pathogenesis. (A) Conversion of fibrinogen to fibrin by prothrombin, Coa.prothrombin or vwb.prothrombin was detected in the presence or absence of 200 ng argatroban (Agb). Arbitrary units are defined as A450*100. Average and standard error of the means were calculated from three independent measurements. (B) Agglutination of S. aureus Newman or S. aureus USA300 LAC in plasma in the presence of increasing concentrations of Agb. Average and standard error of the means were calculated from three independent measurements and statistical significance was assessed with the Student's two-tailed t-test: *P<0.05, **P<0.0001. (C) Survival of cohorts of BALB/c mice (n=15) treated with saline (mock) or dabigatran-etexilate (Dbg) and infected with either S. aureus Newman or the coa/vwb mutant strain. Statistical significance was analyzed with the logrank test: mock vs. Dbg with wt challenge: P<0.0001; mock vs. Dbg with coa/vwb challenge: P=0.43. Data are representative of three independent experiments. (D) Survival of cohorts of BALB/c mice (n=15) treated with saline (mock) or dabigatran (Dbg) and challenged by intravenous inoculation with S. aureus USA300 LAC. Statistical significance was analyzed with the logrank test: mock vs. Dbg, P<0.01. Data are representative of three independent experiments.

FIGS. 7A-7C. Additive protective effects of direct thrombin inhibitors and ClfA-specific antibodies against S. aureus sepsis. (A) Survival of cohorts of BALB/c mice (n=15) treated with saline (mock) or dabigatran (Dbg) followed by intravenous inoculation with S. aureus Newman (wt), clfA or clfA (pClfA) variants. Data are representative of three independent experiments. (B) Survival of cohorts of BALB/c mice (n=15) treated with saline (mock) or Dbg and passively immunized (5 mg·kg−1) with affinity-purified antibodies against V10 or ClfA. Animals were challenged by intravenous inoculation with S. aureus Newman. Statistical analysis was assessed with the logrank test: mock-V 10 vs. mock-ClfA, P<0.001; Dbg-mock vs. Dbg-ClfA, P<0.001. Data are representative of three independent experiments. (C) Half-maximal IgG titer of α-V10 or α-ClfA in serum of passively immunized mice (n=5) was determined by ELISA. Blood was drawn on day 0, six hours post-immunization and at day 10, when the experiment was terminated.

FIGS. 8A-8D. Direct thrombin inhibitors and ClfA-specific antibodies increase the time-to-death of MRSA sepsis in mice. (A) Agglutination of methicillin-resistant S. aureus isolates N315 or MW2 in plasma in the presence or absence of argatroban (Agb). Average and standard error of the means were calculated from at least five independent measurements and statistical significance was assessed with the Student's two-tailed t-test: *P<0.05, ***P<0.0001. (B) Affinity-purified rabbit IgG specific for ClfA (α-ClfA) or the plague protective antigen V10 (α-V10) was analyzed for its ability to prevent agglutination of MRSA strains N315 and MW2 in plasma. Average and standard error of the mean were calculated from 16 fields of view from two independent experiments. Statistical significance of antibody effects compared to a mock treated control was assessed with the Student's two-tailed t test: ***P<0.001. (C) Survival of cohorts of BALB/c mice (n=15) treated with saline (mock) or Dbg and passively immunized (5 mg·kg−1) with affinity-purified antibodies against V10 or ClfA. Animals were challenged by intravenous inoculation with MRSA strain N315. Statistical analysis was assessed with the logrank test: mock-V10 vs. mock-ClfA, not significant; mock-V10 vs. Dbg-ClfA, P<0.05; mock-ClfA vs. Dbg-ClfA, P<0.05; Dbg-mock vs. Dbg-ClfA, P<0.01. Data are representative of two independent experiments. (D) Survival of cohorts of BALB/c mice (n=15) treated as described in panel (C) and challenged by intravenous inoculation with MRSA strain MW2. Statistical analysis was assessed with the logrank test: mock-V10 vs. mock-ClfA, not significant; mock-ClfA vs. Dbg-ClfA, P<0.001; Dbg-mock vs. Dbg-ClfA, P<0.001. Data are representative of two independent experiments.

FIG. 9A-9B. Direct thrombin inhibitors and their effect on in vitro and in vivo coagulation. (A) Conversion of fibrinogen to fibrin by human alpha-thrombin was measured in the presence or absence of 200 ng argatroban. Human prothrombin was incubated alone as negative control. One arbitrary unit is defined as A450*100. Error bars represent standard deviation of triplicate experiments. (B) Dilute thrombin time was measured for plasma from mice treated with saline (mock) or Dabigatran-etexilate (Dbg) on the day of infection or on day 10 following infection. Each symbol represents a blood sample from a single mouse. Horizontal lines indicate mean thrombin time for the cohort. Statistical significance was determined by two-tailed Student's t-test: *P<0.01, **P<0.001.

FIG. 10. Treatment with Dabigatran-etexilate (Dbg) reduces staphylococcal agglutination in heart tissues during sepsis. Summary of histopathology findings in thin-sectioned and hematoxylin-eosin stained heart tissue from BALB/c mice (n=15) treated with saline (mock) or dabigatran-etexilate (Dbg) and infected with S. aureus Newman for 12 hours. Statistical significance was analyzed with the logrank test: mock vs. Dbg. Data are representative of three independent experiments. Representative lesions in heart tissues included staphylococcal agglutination without PMNs (SA), with PMNs (SA+PMNs), and PMN accumulation without staphylococcal agglutination (PMNs−SA). Black bars (Total) represent the sum of all lesions in mock and Dbg treated animals. Error bars represent standard error of the mean from 15 hearts.

### DETAILED DESCRIPTION

Embodiments described herein relate to infection by Staphylococcus, or more precisely, the inhibition of Staphylococcus infection.

### A. PREVENTATIVE AND TREATMENT METHODS

Methods include treatments for a disease or condition caused by a Staphylococcus pathogen by providing or administering agents that inhibit agglutination of Staphylococci. A ClfA inhibitor and a thrombin inhibitor can be given to a person infected with or exposed to Staphylococcus or suspected of having been exposed to staphylococcus or at risk of developing a Staphylococcus infection. Methods may be employed with respect to individuals who have tested positive for exposure to Staphylococcus or who are deemed to be at risk for infection based on possible exposure.

In particular, methods include treatment for Staphylococcal infection. In certain embodiments, the infection includes, but is not limited to pneumonia, sepsis, corneal infection, respiratory infection, skin infection, sinus infection, infection of the central nervous system, or toxic shock syndrome. Staphylococcus infections of the skin that can be treated using the methods and compositions including, but not limited to, dermonecrotic skin infections, eczema, secondary infections associated with eczema, impetigo, ecthyma, cellulitis, folliculitis, psoriasis, boils (furuncles and carbuncles) and sycosis.

In some embodiments, the treatment is administered in conjunction with Staphylococcus antigens or antibodies that bind Staphylococcus bacteria and/or their proteins and/or carbohydrates. Furthermore, in some examples, treatment comprises administration of other agents commonly used against bacterial infection, such as one or more antibiotics.

The compositions and related methods, particularly administration of a ClfA inhibitor and a thrombin inhibitor, may also be used in combination with the administration of traditional antimicrobial therapies. These include, but are not limited to, the administration of vaccines; anti-bacterial antibodies; or antibiotics such as streptomycin, ciprofloxacin, doxycycline, gentamycin, chloramphenicol, trimethoprim, vancomycin, sulfamethoxazole, ampicillin, tetracycline or various combinations of antibiotics.

In one aspect, it is contemplated that therapy includes antibacterial agents other than a ClfA inhibitor or a thrombin inhibitor. Alternatively, the therapy may precede or follow the other agent treatment by intervals ranging from minutes to weeks. In embodiments where the other agents and/or a proteins or polynucleotides are administered separately, one would generally ensure that a significant period of time did not expire between the time of each delivery, such that the agent and antigenic composition would still be able to exert an advantageously combined effect on the subject. In such instances, it is contemplated that one may administer both modalities within about 12-24 h of each other or within about 6-12 h of each other. In some situations, it may be desirable to extend the time period for administration significantly, where several days (2, 3, 4, 5, 6 or 7) to several weeks (1, 2, 3, 4, 5, 6, 7 or 8) lapse between the respective administrations.

Various combinations may be employed, for example, a ClfA inhibitor is “A” and the thrombin inhibitor is “B” or a combination inhibitor therapy is “A” and another antimicrobial agent is “B” and the following regimen is followed:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of these compositions to a patient/subject will follow general protocols for the administration of such compounds, taking into account the toxicity, if any, of the composition, or other compositions described herein. It is expected that the treatment cycles would be repeated as necessary. It also is contemplated that various standard therapies, such as hydration, may be applied in combination with the described therapy.

1. ClfA and ClfA Inhibitors

S. aureus strains express clumping factor A (ClfA) (McDevitt et al., 1994), a surface protein that promotes precipitation of staphylococci through association with soluble fibrinogen (Hawiger et al., 1982) (ClfA is also known as serine-aspartate repeat (Sdr) region). The N2 and N3 domains of ClfA (residues 229-545) bind to the C-terminal end of the fibrinogen γ-chains (residues 395-411) (Ganesh et al., 2008; Strong et al., 1982). S. aureus mutants lacking functional clfA display virulence defects in mouse models for septic arthritis or endocarditis, phenotypes that have been attributed to the loss of staphylococcal binding to fibrinogen deposited on inflamed joint tissues or on mechanically damaged heart valves (Josefsson et al., 2001; Moreillon et al., 1995). ClfA also contributes to staphylococcal escape from phagocytic killing, which involves its binding to complement regulatory factor I (Hair et al., 2010). A ClfA-specific monoclonal antibody has been isolated that blocks staphylococcal association with the fibrinogen γ-chain (Hall et al., 2003). A phase II clinical trial with bacteremic patients compared the efficacy of monoclonal antibody (Tefibazumab) and antibiotic treatment with placebo and antibiotic. However, composite clinical end point analysis did not detect differences (Weems Jr. et al., 2006).

Birch-Hirschfeld employed a biochemical approach to elucidate S. aureus agglutination in citrate-plasma and proposed a reaction pathway involving both fibrinogen and prothrombin (Birch-Hirschfeld, 1934). If so, agglutination must be considerably more complex than the simple association of staphylococci with fibrinogen (clumping).

The Examples below indicate that staphylococcal agglutination in blood is associated with a lethal outcome of S. aureus sepsis in mice. Three secreted products of staphylococci—coagulase (Coa), von Willebrand factor binding protein (vWbp) and clumping factor (ClfA)—are required for agglutination. Coa and vWbp activate prothrombin to cleave fibrinogen, whereas ClfA allowed staphylococci to associate with the resulting fibrin cables. All three virulence genes promoted the formation of thromboembolic lesions in heart tissues. S. aureus agglutination could be disrupted and the lethal outcome of sepsis could be prevented by combining dabigatran-etexilate treatment, which blocked Coa and vWbp activity, with antibodies specific for ClfA. These results provide evidence that the combined administration of direct thrombin inhibitors and ClfA-antibodies that block S. aureus agglutination can be useful for the prevention of staphylococcal sepsis in humans.

ClfA inhibitors include small molecules, nucleic acids (including but not limited to siRNAs, miRNAs, antisense molecules), peptide mimetics, peptides (proteins smaller than 100 amino acids), polypeptides, and proteins (including but not limited to antibodies or antibody fragments).

a. Nucleic Acids

Some embodiments concern polynucleotides or nucleic acid molecules relating to ClfA sequences in diagnostic, therapeutic, and preventative applications. In certain embodiments, a nucleic acid serves as a ClfA inhibitor for the prevention or treatment of Staphylococcus infection and related conditions or diseases. Nucleic acids or polynucleotides of the invention may be DNA or RNA, and they may be olignonucleotides (100 residues or fewer) in certain embodiments. Moreover, they may be recombinantly produced or synthetically produced.

These polynucleotides or nucleic acid molecules may be isolatable and purifiable from cells or they may be synthetically produced. In some embodiments, a ClfA-encoding nucleic acid is the target of a nucleic acid ClfA inhibitor, such as a ribozyme, antisense, miRNA, or siRNA that reduces the level of ClfA expression.

As used in this application, the term “polynucleotide” refers to a nucleic acid molecule, RNA or DNA, that has been isolated free of total genomic nucleic acid. Therefore, a “polynucleotide encoding ClfA” refers to a nucleic acid sequence (RNA or DNA) that contains ClfA coding sequences, yet may be isolated away from, or purified and free of, total genomic DNA and proteins.

The term “cDNA” is intended to refer to DNA prepared using RNA as a template. The advantage of using a cDNA, as opposed to genomic DNA or an RNA transcript is stability and the ability to manipulate the sequence using recombinant DNA technology (See Sambrook, 2001; Ausubel, 1996). There may be times when the full or partial genomic sequence is some. Alternatively, cDNAs may be advantageous because it represents coding regions of a polypeptide and eliminates introns and other regulatory regions. In certain embodiments, nucleic acids are complementary or identical to cDNA encoding sequences, such as a ClfA sequence.

The term “gene” is used for simplicity to refer to a functional protein, polypeptide, or peptide-encoding nucleic acid unit. As will be understood by those in the art, this functional term includes genomic sequences, cDNA sequences, and smaller engineered gene segments that express, or may be adapted to express, proteins, polypeptides, domains, peptides, fusion proteins, and mutants. The nucleic acid molecule hybridizing to a ClfA sequence may comprise a contiguous nucleic acid sequence of the following lengths or at least the following lengths: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 10100, 10200, 10300, 10400, 10500, 10600, 10700, 10800, 10900, 11000, 11100, 11200, 11300, 11400, 11500, 11600, 11700, 11800, 11900, 12000 or more (or any range derivable therein) nucleotides, nucleosides, or base pairs of the ClfA sequence. Such sequences may be identical or complementary to SEQ ID NO:1, and they may encode all or part of SEQ ID NO:2.

Accordingly, sequences that have or have at least or at most 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, and any range derivable therein, of nucleic acids that are identical or complementary to a nucleic acid sequence of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 contiguous bases (or any range derivable therein) of SEQ ID NO:1 is contemplated in some embodiments. They may be used as ClfA inhibitors.

“Isolated substantially away from other coding sequences” means that the gene of interest forms part of the coding region of the nucleic acid segment, and that the segment does not contain large portions of naturally-occurring coding nucleic acid, such as large chromosomal fragments or other functional genes or cDNA coding regions. Of course, this refers to the nucleic acid segment as originally isolated, and does not exclude genes or coding regions later added to the segment by human manipulation.

In some embodiments, a ClfA inhibitor that is a nucleic acid may encode an antisense construct. Antisense methodology takes advantage of the fact that nucleic acids tend to pair with “complementary sequences.” By complementary, it is meant that polynucleotides are those which are capable of base-pairing according to the standard Watson-Crick complementarity rules. Inclusion of less common bases such as inosine, 5-methylcytosine, 6-methyladenine, hypoxanthine and others in hybridizing sequences does not interfere with pairing.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

In certain embodiments, the nucleic acid encodes an interfering RNA or siRNA. RNA interference (also referred to as “RNA-mediated interference” or RNAi) is a mechanism by which gene expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin and Avery, 1999; Montgomery et al., 1998; Sharp and Zamore, 2000; Tabara et al., 1999). Activation of these mechanisms targets mature, dsRNA-complementary mRNA for destruction. Advantages of RNAi include a very high specificity, ease of movement across cell membranes, and prolonged down-regulation of the targeted gene (Fire et al., 1998; Grishok et al., 2000; Ketting et al., 1999; Lin and Avery et al., 1999; Montgomery et al., 1998; Sharp et al., 1999; Sharp and Zamore, 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp et al., 1999; Sharp and Zamore, 2000; Elbashir et al., 2001). It is generally accepted that RNAi acts post-transcriptionally, targeting RNA transcripts for degradation. It appears that both nuclear and cytoplasmic RNA can be targeted (Bosher and Labouesse, 2000).

siRNAs are designed so that they are specific and effective in suppressing the expression of the genes of interest. Methods of selecting the target sequences, i.e., those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998). Alternatively, a naturally occurring miRNA that targets ClfA may be used in some embodiments.

The making of siRNAs has been mainly through direct chemical synthesis; or through an in vitro system derived from S2 cells. Chemical synthesis proceeds by making two single stranded RNA-oligomers followed by the annealing of the two single stranded oligomers into a double-stranded RNA. Methods of chemical synthesis are diverse. Non-limiting examples are provided in U.S. Pat. Nos. 5,889,136, 4,415,723, and 4,458,066, expressly incorporated herein by reference, and in Wincott et al. (1995).

Several further chemical modifications to siRNA or miRNA sequences have been suggested in order to alter their stability or improve their effectiveness. It is suggested that synthetic complementary 21-mer RNAs having di-nucleotide overhangs (i.e., 19 complementary nucleotides +3′ non-complementary dimers) may provide the greatest level of suppression. These protocols primarily use a sequence of two (2′-deoxy)thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang. In other embodiments there is a modification at the 5′ end of the sequence that is complementary to the ClfA target.

In some embodiments, the invention concerns an siRNA that is capable of triggering RNA interference, a process by which a particular RNA sequence is destroyed. siRNA are dsRNA molecules that are 100 bases or fewer in length (or have 100 basepairs or fewer in its complementarity region). In some cases, it has a 2 nucleotide 3′ overhang and a 5′ phosphate. The particular RNA sequence is targeted as a result of the complementarity between the dsRNA and the particular RNA sequence. It will be understood that dsRNA or siRNA of the invention can effect at least a 20, 30, 40, 50, 60, 70, 80, 90 percent or more reduction of expression of a targeted RNA in a cell. dsRNA of the invention (the term “dsRNA” will be understood to include “siRNA”) is distinct and distinguishable from antisense and ribozyme molecules by virtue of the ability to trigger RNAi. Structurally, dsRNA molecules for RNAi differ from antisense and ribozyme molecules in that dsRNA has at least one region of complementarity within the RNA molecule. The complementary (also referred to as “complementarity”) region comprises at least or at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 contiguous bases, or any range derivable therein, to sequences (or their complements) disclosed herein. In some embodiments, the sequence is SEQ ID NO:1. It is specifically contemplated that a dsRNA may be a molecule comprising two separate RNA strands in which one strand has at least one region complementary to a region on the other strand. Alternatively, a dsRNA includes a molecule that is single stranded yet has at least one complementarity region as described above (see Sui et al., 2002 and Brummelkamp et al., 2002 in which a single strand with a hairpin loop is used as a dsRNA for RNAi). For convenience, lengths of dsRNA may be referred to in terms of bases, which simply refers to the length of a single strand or in terms of basepairs, which refers to the length of the complementarity region. It is specifically contemplated that embodiments discussed herein with respect to a dsRNA comprised of two strands are contemplated for use with respect to a dsRNA comprising a single strand, and vice versa. In a two-stranded dsRNA molecule, the strand that has a sequence that is complementary to the targeted mRNA is referred to as the “antisense strand” and the strand with a sequence identical to the targeted mRNA is referred to as the “sense strand.”Similarly, with a dsRNA comprising only a single strand, it is contemplated that the “antisense region” has the sequence complementary to the targeted mRNA, while the “sense region” has the sequence identical to the targeted mRNA. Furthermore, it will be understood that sense and antisense region, like sense and antisense strands, are complementary (i.e., can specifically hybridize) to each other.

The single RNA strand or two complementary double strands of a dsRNA molecule may be of at least or at most the following lengths: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 31, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 or more (including the full-length of a particular's gene's mRNA without the poly-A tail) bases or basepairs. If the dsRNA is composed of two separate strands, the two strands may be the same length or different lengths. If the dsRNA is a single strand, in addition to the complementarity region, the strand may have 1, 2, 3, 4, 5, 6, 7, 8, 9, 10, 11, 12, 13, 14, 15, 16, 17, 18, 19, 20, 21, 22, 23, 24, 25, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more bases on either or both ends (5′ and/or 3′) or as forming a hairpin loop between the complementarity regions.

b. Proteins and Polypeptides

Embodiments concern methods and compositions involving a ClfA inhibitor that is a polypeptide. In certain embodiments, the ClfA polypeptide inhibitors are used in the treatment or prevention of Staphylococcus infection and conditions and diseases that are caused by such infection. The terms “protein” and “polypeptide” are used interchangeably herein and they both cover what is understood as a “peptide” (a polypeptide molecule having 100 or fewer amino acid residues). In certain embodiments of the present invention, the ClfA inhibitor is a protein, polypeptide, or peptide; in particular embodiments, the ClfA inhibitor is protein or polypeptide that is an antibody.

In some embodiments, the ClfA inhibitor is a peptide, which is a polypeptide that is less than 100 amino acids in length. In certain embodiments, the ClfA inhibitor is a peptide that is 1, 2, 3, 4, 5, 6, 7, 8, 9, 10, 11, 12, 13, 14, 15, 16, 17, 18, 19, 20, 21, 22, 23, 24, 25, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 amino acids (or any range derivable therein, in length. In particular embodiments the peptide is 7 residue peptide as described in Strong et al. and Ganesh et al., which are both incorporated by reference.

As will be understood by those of skill in the art, modification and changes may be made in the structure of a ClfA inhibitor polypeptide or peptide, and still produce a molecule having like or otherwise desirable characteristics. For example, certain amino acids may be substituted for other amino acids or include deletions, additions, or truncations in the protein sequence without appreciable loss of interactive binding capacity with structures. Since it is the interactive capacity and nature of a protein that defines that protein's biological functional activity, certain amino acid sequence substitutions can be made in a protein sequence (or, of course, its underlying DNA coding sequence) and nevertheless obtain a protein with similar inhibitory properties. It is thus contemplated by the inventors that various changes may be made in the sequence of ClfA inhibitor polypeptides or peptides (or underlying DNA) without appreciable loss of their biological utility or activity.

It is also well understood that where certain residues are shown to be particularly important to the biological or structural properties of a protein or peptide, e.g., residues in the binding site of an antibody, such residues may not generally be exchanged.

Amino acid substitutions are generally based on the relative similarity of the amino acid side-chain substituents, for example, their hydrophobicity, hydrophilicity, charge, size, and the like. An analysis of the size, shape, and type of the amino acid side-chain substituents reveals that arginine, lysine, and histidine are all positively charged residues; that alanine, glycine, and serine are all a similar size; and that phenylalanine, tryptophan, and tyrosine all have a generally similar shape. Therefore, based upon these considerations, the following subsets are defined herein as biologically functional equivalents: arginine, lysine, and histidine; alanine, glycine, and serine; and phenylalanine, tryptophan, and tyrosine.

To effect more quantitative changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle, 1982, incorporated herein by reference). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within ±1 are particularly preferred, some, and those within ±0.5 are even more particularly preferred.

It is also understood in the art that the substitution of like amino acids can be made effectively on the basis of hydrophilicity, particularly where the biological functional equivalent protein or peptide thereby created is intended for use in immunological embodiments, as in the present case. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±2, ±1, or ±0.5 is contemplated.

Some embodiments pertain to methods and compositions involving an inhibitor of ClfA, wherein the inhibitor is an antibody that binds ClfA. In addition to the ClfA antibodies discussed below, other ClfA antibodies are available. These include mouse monoclonal antibodies, as well as chimeric and humanized versions such as those found in U.S. Pat. No. 6,979,446, which is hereby incorporated by reference. In some embodiments, it is specifically contemplated that a method or composition may include an antibody that contains sequences from any of the antibodies known as 12-9, 13-2, 13-1, 35-220, 35-006, 12-9A, and 35-052.1 in U.S. Pat. No. 6,979,446, which is hereby incorporated by reference. ClfA antibodies may also be any of those set forth in U.S. Patent Publication 20110020323 (referred to as SdrA antibodies), which is specifically incorporated by reference. An example includes the rG1 antibody, and derivatives thereof, which may be included in compositions and methods described herein.

As used herein, the term “antibody” refers to any form of antibody or fragment thereof that exhibits the desired biological activity. Thus, it is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity—that is inhibition of ClfA activity. An antibody inhibitor may be considered a neutralizing antibody.

Included within the definition of an antibody that binds ClfA is a ClfA antibody binding fragment. As used herein, the term “ClfA binding fragment” or “binding fragment thereof” encompasses a fragment or a derivative of an antibody that still substantially retain its biological activity of inhibiting ClfA activity. Therefore, the term “antibody fragment” or ClfA binding fragment refers to a portion of a full length antibody, generally the antigen binding or variable region thereof. Examples of antibody fragments include Fab, Fab′, F(ab′)2, and Fv fragments; diabodies; linear antibodies; single-chain antibody molecules, e.g., sc-Fv; and multispecific antibodies formed from antibody fragments. Typically, a binding fragment or derivative retains at least 50% of its ClfA inhibitory activity. Preferably, a binding fragment or derivative retains about or at least about 60%, 70%, 80%, 90%, 95%, 99% or 100% of its ClfA inhibitory activity. It is also intended that a ClfA binding fragment can include conservative amino acid substitutions that do not substantially alter its biologic activity.

The term “monoclonal antibody”, as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975), or may be made by recombinant DNA methods (see, e.g., U.S. Pat. No. 4,816,567). The “monoclonal antibodies” may also be isolated from phage antibody libraries using the techniques described in Clackson et al. (1991) and Marks et al. (1991), for example. A monoclonal antibody may be a mouse antibody or it may be a human antibody.

As used herein, the term “humanized antibody” refers to forms of antibodies that contain sequences from non-human (e.g., murine) antibodies as well as human antibodies. Such antibodies are chimeric antibodies which contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Any suitable method for generating monoclonal antibodies may be used. For example, a recipient may be immunized with ClfA or a fragment thereof. Any suitable method of immunization can be used. Such methods can include adjuvants, other immunostimulants, repeated booster immunizations, and the use of one or more immunization routes.

Any suitable source of ClfA can be used as the immunogen for the generation of the non-human antibody of the compositions and methods disclosed herein. Such forms include, but are not limited whole protein, peptide(s), and epitopes, generated through recombinant, synthetic, chemical or enzymatic degradation means known in the art.

Any form of the ClfA antigen can be used to generate the antibody that is sufficient to generate a biologically active antibody. In some embodiments, the antigen or a ClfA fragment comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 710, 720, 730, 740, 750, 760, 770, 780, 790, 800, 810, 820, 830, 840, 850, 860, 870, 880, 890, 900, 910, 920, 930, or 933 contiguous amino acids, or any range derivable therein, of SEQ ID NO:2. Alternatively, the antigen may be 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any range derivable therein, identical to SEQ ID NO:2 or to a fragment thereof.

Thus, the eliciting antigen may be a single epitope, multiple epitopes, or the entire protein alone or in combination with one or more immunogenicity enhancing agents known in the art. The eliciting antigen may be an isolated full-length protein, a cell surface protein (e.g., immunizing with cells transfected with at least a portion of the antigen), or a soluble protein (e.g., immunizing with only the extracellular domain portion of the protein). The antigen may be produced in a genetically modified cell. The DNA encoding the antigen may genomic or non-genomic (e.g., cDNA) and encodes at least a portion of the extracellular domain. As used herein, the term “portion” refers to the minimal number of amino acids or nucleic acids, as appropriate, to constitute an immunogenic epitope of the antigen of interest. Any genetic vectors suitable for transformation of the cells of interest may be employed, including but not limited to adenoviral vectors, plasmids, and non-viral vectors, such as cationic lipids.

c. Small Molecules

Some embodiments concern ClfA inhibitors that are small molecules, which refers to a small compound that is biologically active but is not a polymer. It does refer to a monomer.

2. Thrombin Inhibitors

In some embodiments, a ClfA inhibitor works in conjunction with a thrombin inhibitor. Thrombin inhibitors are compounds that inhibit coagulation by directly inhibiting thrombin. Blood clotting may be evaluated in a patient to establish a baseline level of activity and/or it may be evaluated after the patient has undergone some thrombin inhibitor therapy. Examples of thrombin inhibitors are described throughout this disclosure; they include, but are not limited to, argatroban, melagratran (or its prodrug ximelagatran), dabigatran, efegatran, and inogatran.

The ecarin clotting time is the appropriate monitoring test in some embodiments. In other embodiments, activated partial thromboplastin over time may be employed to evaluate anti-thrombin activity. Other tests are well known to those of skill in the art.

Other related embodiments can be found in the Examples below. This test may be performed and the results may be provided to a clinician who can decide whether to place a patient on a particular therapy, continue a patient on a particular therapy, or change the particular drug to implement the therapy.

Examples of univalent direct thrombin inhibitors include Argatroban, Ximelagatran, and Dabigatran etexilate. Some possible dosage regimens are provided below.

With Argatroban, a patient may receive an initial dose of 1.9-2.1 μg/kg/min (though in some embodiments, the initial dose is lower) and a final dose of 1.6 (0.25-4.0) μg/kg/min; during a median of 6 days of argatroban therapy, the patient may undergo 0, 1, 2, 3, 4, or 5 dosage adjustments using a median and mode incremental adjustment of 0.5 μg/kg/min (5-95th percentile, 0.1-2.0 μg/kg/min). Incremental adjustments may decrease with decreasing current dose (e.g., median 0.25 μg/kg/min from doses of 0.26-0.75 μg/kg/min) See Verme-Gibboney et al., 2003, which is hereby incorporated by reference.

In other embodiments involving Argabatron, a bolus of 100 μg/kg of argatroban is given over 1 min followed by an infusion of either 1.0 μg/kg/min or 3.0 μg/kg/min. In another embodiment, Argatroban therapy is initiated at 2 μg kg per minute. A lower starting dose is possible because of the patient's medical condition, such as hepatic impairment. The aPTT may be determined 1-2 hours later, and the dose may be adjusted (up to 10 μg kg per minute maximum) until the aPTT is 1.5 to 3 times the baseline aPTT value (not to exceed 100 seconds). Patients with HIT may receive argatroban at a mean (SD) dose of 1.7 (1.0) μg kg per minute over a mean (SD) duration of 5.1 (4.2) days.

For therapy involving Melagatran, in one embodiment a patient receives single s.c. doses of melagatran (0.1-5 mg); in other embodiments, 3 mg s.c. melagatran is administered at 12-h intervals for 4 days (toxicity began at doses over 5 mg).

In additional embodiments, Ximelagatran may be dosed twice daily. Oral ximelagatran 36 mg twice daily may be employed. In other embodiments, there may be a daily dose of 24 mg or 36 mg with treatment continuing for 7 to 12 days. Alternatively, therapy may involve a single dose of subcutaneous melagatran 2 mg followed by melagatran 3 mg subcutaneously after surgery and then oral ximelagatran 24 mg twice daily for a total treatment duration of 8 to 11 days. In certain embodiments, the initial dose of melagatran may be administered prior to an invasive medical procedure such a surgery.

In some embodiments, Dabigatran etexilate is administered in amounts at or above 12.5 mg and at or below 300 mg twice daily.

In some embodiments, efegatran sulphate is provided to a patient at levels of at least 0.63 mg/kg/hr to produce anti-thrombotic effect that is at least comparable to an activated partial thromboplastin time adjusted heparin infusion.

In further embodiments, Efegatran may be provided to a patient at a dose level of 1.2 mg/kg/hr resulting in steady state mean activated partial thromboplastin time values of approximately three times baseline. A range of efegatran doses includes 0.63 to 1.2 mg/kg/hour.

3. General Pharmaceutical Compositions

In some embodiments, pharmaceutical compositions are administered to a subject. Different embodiments involve administering an effective amount of a composition to a subject. In some embodiments of the present invention, a composition comprising a ClfA inhibitor and thrombin inhibitor may be administered to the subject or patient to protect against or treat infection by one or more Staphylococcus pathogens. Additionally, such compounds can be administered in combination with an antibiotic or another standard antibacterial therapy. Such compositions will generally be dissolved or dispersed in a pharmaceutically acceptable carrier or aqueous medium.

The active compounds described herein can be formulated for parenteral administration, e.g., formulated for injection via the intravenous, intramuscular, sub-cutaneous, or even intraperitoneal routes. The preparation of an aqueous composition that contains a compound or compounds that inhibit thrombin and/or ClfA will be known to those of skill in the art in light of the present disclosure. Typically, such compositions can be prepared as injectables, either as liquid solutions or suspensions; solid forms suitable for use to prepare solutions or suspensions upon the addition of a liquid prior to injection can also be prepared; and, the preparations can also be emulsified. In addition to the compounds formulated for parenteral administration, other pharmaceutically acceptable forms include, e.g., aerosolizable, inhalable, or instillable formulations; tablets or other solids for oral administration; time release capsules; creams; lotions; mouthwashes; and the like. The preparation of an such formulations will be known to those of skill in the art in light of the present disclosure.

In some embodiments, a formulation provides for the extended release of an active component.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable carrier,” means a pharmaceutically acceptable material, composition or vehicle, such as a liquid or solid filler, diluent, excipient, solvent or encapsulating material, involved in carrying or transporting a chemical agent. The proteinaceous compositions may be formulated into a neutral or salt form. Pharmaceutically acceptable salts include the acid addition salts (formed with the free amino groups of the protein) and those which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Such compositions would normally be administered as pharmaceutically acceptable compositions that include physiologically acceptable carriers, buffers or other excipients.

For parenteral administration in an aqueous solution, for example, the solution should be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media which can be employed will be known to those of skill in the art in light of the present disclosure. For example, one dosage could be dissolved in isotonic NaCl solution and either added to hypodermoclysis fluid or injected at the proposed site of infusion, (see for example, Remington's Pharmaceutical Sciences, 1990). Some variation in dosage will necessarily occur depending on the condition of the subject. The person responsible for administration will, in any event, determine the appropriate dose for the individual subject.

Some variation in dosage will necessarily occur depending on the condition of the subject. The person responsible for administration will, in any event, determine the appropriate dose for the individual subject. An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the effects desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Upon formulation, solutions will be administered in a manner compatible with the dosage formulation and in such amount as is therapeutically or prophylactically effective. The formulations are easily administered in a variety of dosage forms, such as the type of injectable solutions described above.

Typically, for a human adult (weighing approximately 70 kilograms), from about 0.1 mg to about 3000 mg (including all values and ranges there between), or from about 5 mg to about 1000 mg (including all values and ranges there between), or from about 10 mg to about 100 mg (including all values and ranges there between), of a compound are administered. It is understood that these dosage ranges are by way of example only, and that administration can be adjusted depending on the factors known to the skilled artisan.

In certain embodiments, a subject is administered about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 milligrams (mg) or micrograms (mcg or μg) or μg/kg or micrograms/kg/minute or mg/kg/min or micrograms/kg/hour or mg/kg/hour, or any range derivable therein.

It is contemplated that compositions of the invention may be administered to a patient within about 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, and/or 1, 2, 3, 4, 5, 6, 7, 8, 9, 10, 11, 12 months of being diagnosed with infection by Staphylococcus or of being at risk for Staphylococcus infection (such as by being subject to an invasive medical procedure or about to be subject to an invasive medical procedure), or identified as having symptoms of Staphylococcus infection.

In certain embodiments, a course of treatment with both ClfA inhibitor and a thrombin inhibitor will last 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 days or more. It is contemplated that one agent may be given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, any combination thereof, and another agent is given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, or any combination thereof. Within a single day (24-hour period), the patient may be given one or multiple administrations of the agent(s). Moreover, after a course of treatment, it is contemplated that there is a period of time at which no other treatment is administered. This time period may last 1, 2, 3, 4, 5, 6, 7 days, and/or 1, 2, 3, 4, 5 weeks, and/or 1, 2, 3, 4, 5, 6, 7, 8, 9, 10, 11, 12 months or more, depending on the condition of the patient, such as their prognosis, strength, health, etc.

In particular embodiments, compositions containing one or more inhibitors may be administered 1, 2, 3, 4, 5, 6, 7, 8, 9, 10, 11, 12, 13, 14, 15, 16, 17, 18, 19, 20 or more times, and/or they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months, or any range or combination derivable therein.

Another aspect of embodiments described herein is the administration of other therapies or vaccines in conjunction with a ClfA inhibitor and a thrombin inhibitor. Methods of administering immunoglobulins directed at bacterial antigens to a recipient to prevent a staphylococcal infection can be considered a passive vaccine. Another aspect includes the use of active vaccines against staphylococcal infection in conjunction with inhibitors. Certain therapeutic methods include the administration of a therapeutic immunoglobulin or an antigen to stimulate or induce production of an immune response in a subject. A method of preparing an immunoglobulin for use in prevention or treatment of staphylococcal infection comprises the steps of immunizing a recipient or donor with a vaccine and isolating immunoglobulin from the recipient or donor. In certain aspects an immunoglobulin can bind to a fibrinogen, fibrin, or ClfA. A pharmaceutical composition comprising an immunoglobulin, with or without other inhibitors, and a pharmaceutically acceptable carrier can be used in the manufacture of a medicament for the treatment or prevention of staphylococcal disease. A method for treatment or prevention of staphylococcal infection comprising a step of administering to a patient an effective amount of the pharmaceutical preparation of the invention is a further aspect.

The antibodies can be isolated to the extent desired by well known techniques such as affinity chromatography (Harlow and Lane, 1988). Antibodies can include antiserum preparations from a variety of commonly used animals, e.g. goats, primates, donkeys, swine, horses, guinea pigs, rats or man.

Any immunoglobulin, whether directed at any bacterial antigen and produced in accordance with the embodiments discussed herein can include whole antibodies, antibody fragments or subfragments. Antibodies can be whole immunoglobulins of any class (e.g., IgG, IgM, IgA, IgD or IgE), chimeric antibodies or hybrid antibodies with dual specificity to two or more antigens of the invention. They may also be fragments (e.g., F(ab′)2, Fab′, Fab, Fv and the like) including hybrid fragments. An immunoglobulin also includes natural, synthetic, or genetically engineered proteins that act like an antibody by binding to specific antigens to form a complex. In the case of an immunoglobulin directed at a component of agglutination, for example, ClfA, the immunoglobulin may bind to the ClfA and inhibit its activation or activity.

A vaccine can be administered to a recipient who then acts as a source of immunoglobulin, produced in response to challenge from the specific vaccine. A subject thus treated would donate plasma from which hyperimmune globulin would be obtained via conventional plasma fractionation methodology. The hyperimmune globulin would be administered to another subject in order to impart resistance against or treat staphylococcal infection. Hyperimmune globulins of the invention are particularly useful for treatment or prevention of staphylococcal disease in infants, immune compromised individuals, or where treatment is required and there is no time for the individual to produce antibodies in response to vaccination.

### B. EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of certain embodiments, are provided as an example, and are not intended as limitations on the scope of the invention. Changes therein and other uses which are encompassed within the spirit of the invention as defined by the scope of the claims will occur to those skilled in the art.

### Example 1

### Materials and Methods for Examples 2

Animal experiments. Animal experiments involving S. aureus challenge followed protocols that were reviewed, approved and performed under the regulatory supervision of The University of Chicago's Institutional Biosafety Committee (IBC) and the Institutional Animal Care and Use Committee (IACUC). Animals were managed by the University of Chicago Animal Resource Center, which is accredited by the American Association for Accreditation of Laboratory Animal Care and the Department of Health and Human Services (DHHS number A3523-01). Animals were maintained in accordance with the applicable portions of the Animal Welfare Act and the DHHS “Guide for the Care and Use of Laboratory Animals”. Veterinary Care was under the direction of full-time resident veterinarians boarded by the American College of Laboratory Animal Medicine. BALB/c mice and New Zealand white rabbits were purchased from Charles River Laboratories and Harlan Sprague Dawley, respectively. After confirming that the data sets abide by a normal distribution, the statistical analysis of staphylococcal sepsis was analyzed using the two-tailed logrank test. Quantification of mouse heart tissue histopathology was analyzed for statistical significance using the unpaired two-tailed Student's t-test. The bacterial load (CFU) in heart tissue from mice infected with staphylococcal variants was analyzed with the Mann Whitney test. The results of all animal experiments were examined for reproducibility.

Bacterial strains and growth of cultures. S. aureus strains Newman (Baba et al., 2007), USA300 LAC (Diep et al., 2006), MW2 (Baba et al., 2002) and N315 (Kuroda et al., 2001) were cultured on tryptic soy agar or broth at 37° C. E. coli strains DH5a and BL21 (DE3) were cultured on Luria Bertani agar or broth at 37° C. Ampicillin (100 ng/ml) and chloramphenicol (10 ng/ml) were used for pET15b and pOS1 selection (Schneewind et al., 1993), respectively.

Transposon Mutants and Plasmids.

Insertional mutations carrying the bursa aurealis transposon with an erthyromycin resistance cassette from the Phoenix library (Bae et al., 2004) were transduced with bacteriophage into S. aureus Newman or the coa/vwb mutant (Cheng et al., 2010). Mutations were verified by PCR with specific primer pairs for

coa

CGCGGATCCATAGTAACAAAGGATTATAGTGGGAAATCACAAG (SEQ ID NO:3) and TCCCCCGGGTTATTTTGTTACTCTAGGCCCATATGTCGC (SEQ ID NO:4)

vwb

CGCGGATCCGTGGTTTCTGGGGAGAAGAATCC (SEQ ID NO:5) and TCCCCCGGGTTTGCAGCCATGCATTAATTATTTGCC (SEQ ID NO:6) and

clfA

CGCGGATCC-AAGGTCAAATCGACCGTT (SEQ ID NO:7) and CGGGGTACC-TTATTTCTTATCTTTATTTTCTTTTTTTC (SEQ ID NO:8) as well as by immunoblotting with specific rabbit antibodies (Cheng et al., 2010; Stranger-Jones et al., 2006). Complementing plasmids pcoa-vWbp and pclfA were described previously (Cheng et al., 2010; DeDent et al., 2008). For immunoblot analysis, 1 mL of staphylococcal overnight cultures grown in tryptic soy broth (Difco) were centrifuged at 8,000×g for 3 min in a table top centrifuge and the supernatant was recovered. Proteins in culture supernatants were precipitated with 10% trichloroacetic acid on ice for 20 minutes. Pellets were washed once in 1 mL TSM (100 mM Tris-HCl, pH 7.5, 0.5 M sucrose, 10 mM MgCl2), suspended in 500 TSM, incubated with 50 rig lysostaphin for 15 minutes at 37° C. for 15 minutes. 10% TCA was added and samples were incubated on ice for 10 min. All samples were centrifuged and washed with 1 mL ice-cold 100% acetone. Samples were air dried and solubilized in 75 sample buffer (4% SDS, 50 mM Tris-HCl, pH 8.0, 10% glycerol, and bromophenol blue).

Scanning electron microscopy. Staphylococcal strains were grown to mid-log phase (OD600 0.5), washed twice and suspended in PBS to a final OD600 1. Bacteria were mixed with EDTA-chelated rabbit plasma (1:1) and incubated for 15 minutes. Samples were fixed for 60 minutes in 2% glutaraldehyde in phosphate buffered saline (PBS) at room temperature onto freshly prepared poly-L-lysine coated glass coverslips. Samples were washed twice with PBS and subsequently serially dehydrated by consecutive incubations in 25% and 50% ethanol/PBS, 75% and 90% ethanol/H2O, 2×100% ethanol, followed by 50% ethanol/hexamethyldisilazane (HDMS) and finally with 100% HDMS. After overnight evaporation of HDMS at room temperature, samples were mounted onto specimen mounts (Ted Pella, Inc.) and coated with 80% Pt/20% Pd to 8 nm using a Cressington 208HR Sputter Coater at 20 mA prior to examination with a Fei Nova NanoSEM 200 scanning electron microscope. The SEM was operated with an acceleration voltage of 5 kV and samples were viewed at a distance of 5 mm.

Protein purification. E. coli BL21(DE3) harboring expression vectors containing coa, vwb, or clfA were grown at 37° C. and induced with 1 mM IPTG after two hours. Three hours following induction, cells were centrifuged at 7,000×g, suspended in column buffer (0.1 M Tris-HCl, pH 7.5, 0.5 M NaCl) and lysed in a French pressure cell at 14,000 lb/in2. Lysates were subjected to ultracentrifugation at 40,000×g for 30 min and the supernatant was subjected to Ni-NTA chromatography, washed with column buffer containing 10 mM imidazole, followed by elution with 500 mM imidazole. Eluates were dialyzed against PBS. To remove endotoxin, 1:100 Triton-X114 was added and the solution was chilled for 10 min, incubated at 37° C. for 10 min, and centrifuged at 13,000×g. This was repeated twice. Supernatant was loaded onto a HiTrap desalting column to remove remnants of Triton-X114. Purity was verified by SDS-PAGE analysis and Coomassie Brilliant Blue staining.

Rabbit antibodies. Protein concentration was determined using a BCA kit (Pierce). Purity was verified by SDS-PAGE analysis and Coomassie Brilliant Blue staining. Six month old New-Zealand white female rabbits were immunized with 500 μg protein emulsified in CFA (Difco) for initial immunization or IFA for booster immunizations on day 24 and 48. On day 60, rabbits were bled and serum recovered for immunoblotting or passive transfer experiments. For antibody purification, recombinant His6-Coa (Cheng et al., 2010), His6-vWbp (Cheng et al., 2010), or His6-ClfA (5 mg) (Strange-Jones et al., 2006) was covalently linked to HiTrap NHS-activated HP columns (GE Healthcare). This antigen-matrix was then used for affinity chromatography of 10-20 ml of rabbit serum raised against Coa (Cheng et al., 2010), vWbp (Cheng et al., 2010) or ClfA at 4° C. Charged matrix was washed with 50 column volumes of PBS, antibodies eluted with elution buffer (1 M glycine pH 2.5, 0.5 M NaCl) and immediately neutralized with 1 M Tris-HCl, pH 8.5. Purified antibodies were dialyzed overnight against PBS, 0.5 M NaCl at 4° C.

Agglutination assay. Overnight cultures of staphylococcal strains were washed in 1 mL 0.85% NaCl and suspended to a final concentration of OD600 4.0 in 1 mL. Bacteria were incubated with 1:500 Syto9 (Invitrogen) for 15 minutes, washed with 1 mL 0.85% NaCl, and suspended in 1 mL saline. Bacteria were mixed 1:1 with EDTA-chelated rabbit plasma (Becton, Dickinson) on a glass microscope slide and incubated for 15 minutes. Samples were viewed and images captured on an Olympus Provis microscope using a 40× objective. For quantification of agglutination, plasma and bacteria were inoculated onto polystyrene C-Chip disposable hemocytometer slides (IN-CYTO). Brightfield images from sixteen fields of view were taken of bacterial strains using a Nikon TE2000U with a 20× objective. To determine the degree of agglutination, 12-20 random fields of were viewed and fluorescent micrographs acquired. For analysis, the largest aggregate in each field of view was outlined in ImageJ and the area of that aggregate measured. Mean area of S. aureus aggregates were determined and normalized by subtracting Newman in saline without plasma from all values. Percent agglutination was calculated by normalizing all mean intensity values to S. aureus Newman in plasma. To assess the inhibitory affect of antibodies on agglutination, affinity-purified polyclonal antibodies were incubated with staphylococci to a final concentration of 3 μM for 10 minutes prior to mixture with plasma. Fold change in agglutination was calculated as the ratio of mean bacterial aggregation area in the presence of antibody compared to control. Statistical significance, P<0.05, was determined by one way analysis of variance (ANOVA), with Dunnett's multiple comparison analysis for post-hoc testing using GraphPad Prism software. To assess the inhibitory affect of argatroban on agglutination, argatroban was diluted 1:10 and 1:100 in plasma and incubated for 10 minutes prior to mixture with bacteria. Percent agglutination was measured compared to bacteria in plasma without argatroban. For experiments using S. aureus N315 and MW2, agglutination was measured as percent change in OD550 following two hours incubation of bacteria with saline containing argatroban (1 mg/mL), plasma, or plasma containing argatroban (1 mg/mL). Error bars represent standard error of the mean from at least three independent experiments to ensure reproducibility.

Sepsis. Overnight cultures of staphylococcal strains were diluted 1:100 into fresh TSB and grown until they reached an OD600 of 0.4. Bacteria were centrifuged at 7,000×g, washed, and suspended in the one-tenth volume of PBS. Six week-old female BALB/c mice (n=15) (Charles River) were injected retro-orbitally with 1×108 CFU (S. aureus Newman, MW2, and N315) or 5×107 CFU (S. aureus USA300) suspensions in 100 μl of PBS. Mice were monitored for survival over 10 days. To enumerate staphylococcal load in heart tissue twelve hours post-infection, mice were euthanized by CO2 asphyxyation and hearts were removed during necropsy. Heart tissue was homogenized in PBS, 0.1% Triton X-100. Serial dilutions of homogenate were spread on TSA and incubated for colony formation. The bacterial load in organ tissue was analyzed in pairwise comparisons between wild-type and mutant strains with the unpaired two-tailed Student's t-test. For histopathology, mice infected with S. aureus were euthanized 12 hours after infection. Hearts were removed during necropsy and fixed in 10% formalin for 24 hours at room temperature. Tissues were embedded in paraffin, thin-sectioned, stained with hematoxylin and eosin, and examined by light microscopy to enumerate pathological lesions per organ. Data were analyzed in pairwise comparisons between wild-type and mutant strains with the unpaired two-tailed Student's t-test. For immunohistochemical analysis, thin-sectioned heart tissues were stained with polyclonal antibodies against mouse prothrombin (Haematologic Technologies) or mouse fibrinogen (Haematologic Technologies).

ClfA binding to fibrinogen and fibrin. MaxSorb 96-well ELISA plates (Nunc) were coated with human fibrinogen (Sigma) overnight. Wells were washed and solutions of PBS or alpha-thrombin (Innovative Research), 100 nM in 1% sodium-citrate/PBS were added for one hour at room temperature to generate fibrinogen and fibrin wells respectively. As controls, the same conditions were generated in Eppendorf tubes. Following incubation with or without alpha-thrombin, samples were centrifuged at 13,000×g for 10 min and supernatants were recovered. The sediment was dissolved in 8 M urea. Running buffer (3 M urea, 4% SDS, 10% BME) was added 1:1. Proteins in supernatants and pellets were separated by SDS-PAGE (15%) and stained with Coomassie Brilliant Blue to analyze soluble fibrinogen in the supernatant fraction and fibrin in the sediment. Purified recombinant ClfA in 1% sodium-citrate was added at increasing concentrations to 96-well plates and incubated for one hour. Samples were incubated with polyclonal anti-ClfA (1:1,000) to detect bound-ClfA followed by goat anti-rabbit-HRP (1:10,000). The wells were developed using an OptEIA kit (BD Lifesciences) and absorbance at 450 nm was measured. Non-linear regression assuming one-site saturation kinetics was performed using GraphPad Prism.

Active immunization. Three week-old BALB/c mice (n=10) were injected with 50 μg protein emulsified in 100 μl complete Freund's adjuvant. Eleven days post vaccination these mice were boosted with 50 μg protein each emulsified in 100 μl incomplete Freund's adjuvant. On day 21, mice were injected with 1×108 CFU of S. aureus challenge strains.

Passive transfer of antibodies. Six hours prior to infection, six week old BALB/c mice (n=15) were injected intraperitoneally with antibody doses of 5 mg/kg body weight. Polyclonal antibodies were affinity purified on antigen-coupled resin and affinity purified V10 (LcrV plague antigen) rabbit antibodies were used as control. Control mice (n=5) that received the same antibody via passive transfer were anesthetized and bled retro-orbitally at the time of infection and again at the end of the experiment. Blood was collected using micro-hematocrit capillary tubes (Fisher) in Z-Gel microtubes (Sarstedt). Tubes were centrifuged at 8,000×g for three minutes, and serum was collected. Antibody titer was measured by ELISA as previously described (Kim et al., 2010). Time to death statistical significance was assessed using the Logrank Test. P<0.01.

Coagulase activity. Purified recombinant Coa or vWbp (100 nM) were mixed with human prothrombin (Innovative Research) in 1% sodium-citrate/PBS. After an initial reading, fibrinogen (3 μM) (Sigma) was added and conversion of fibrinogen to fibrin was measured as an increase in turbidity at 450 nm in a plate reader (BioTek) at 2.5 min intervals. As controls, the enzymatic activity of human alpha-thrombin (Innovative Research) or prothombin alone were measured. Argatroban (200 ng, Novaplus) was added to reactions prior to the addition of fibrinogen.

Dabigatran etexilate treatment. Dabigatran (Boehringer Ingelheim) tablets were dissolved in 0.9 N saline and doses of 10 mg/kg in 100 μL were administered. Mice (n=15) were injected intraperitoneally starting 24 hours prior to infection and continuing every twelve hours during the course of the infection. Control mice received injections of 0.9 N saline. To measure dilute thrombin time, mice (n=5) received saline or dabigatran treatment and were euthanized by CO2 asphyxiation at the time of infection. Blood was drawn by cardiac puncture, diluted in sodium-citrate (1%), centrifuged at 1,500×g for 5 minutes, and plasma diluted in pooled fresh human plasma 1:6. Thrombin time was measured on a STA-R analyzer (Diagnostica Stago).

### Example 2

### Surface Proteins Contribute to Staphylococcal Sepsis

The inventors previously developed an animal model to examine the genetic requirements for staphylococcal sepsis (Kim et al., 2010). Briefly, S. aureus Newman, 1×108 CFU, is injected into the retro-orbital plexus of BALB/c mice, resulting in 100% lethality over a ten day observation period (Kim et al., 2010). This model was used to examine the contribution of secreted coagulases to staphylococcal sepsis (Cheng et al., 2010). S. aureus Newman mutants lacking the coa and vwb genes displayed increased time-to-death and increased survival phenotypes (Cheng et al., 2010) (Table 1). Earlier work identified sortase A (SrtA), an enzyme that links surface proteins to the staphylococcal cell wall envelope (Mazmanian et al., 1999), as an essential virulence factor for sepsis (Kim et al., 2010). Nevertheless, these studies left unresolved which surface protein(s) play a key role in this disease process. S. aureus mutants with insertional lesions in any one of eighteen surface protein genes (Bae et al., 2004) were tested for their role in sepsis (Table 1). These experiments identified clumping factor A (ClfA) as the single most important contributor (Table 1). Although mutations in clfA diminished the severity of clinical disease and improved the outcome of sepsis, clfA mutants retained significant virulence and were still capable of killing infected animals, unlike srtA variants (Table 1).

TABLE 1
Surface protein genes and their contribution to S. aureus sepsis

| | | Median survival time |
|---|---|---|
| Genotype | P values | (hours ± SEM) |
| wild-type | — | 24 (1.6) |
| srtA | <0.0001 | >240 |
| sasF | 1.000 | 24 (1.6) |
| sdrC | 0.5416 | 24 (1.2) |
| sdrD | 0.5416 | 24 (1.2) |
| sasD | 0.3415 | 24 (2.0) |
| isdA | 0.3116 | 24 (1.8) |
| sasG | 0.1462 | 24 (0) |
| clfB | 0.0888 | 24 (1.2) |
| sdrE | 0.0888 | 24 (4.8) |
| isdH | 0.0143 | 24 (2.0) |
| isdB | 0.0243 | 30 (3.2) |
| sasA | 0.0004 | 36 (7.3) |
| isdC | <0.0001 | 36 (1.2) |
| vwb | <0.0001 | 36 (2.6) |
| fnbpA | 0.0004 | 48 (5.5) |
| sasB | <0.0001 | 48 (7.4) |
| sasC | 0.0011 | 54 (8.8) |
| fnbpB | <0.0001 | 60 (8.0) |
| coa | <0.0001 | 72 (12.5) |
| adsA | <0.0001 | 96 (16.7) |
| clfA | <0.0001 | 120 (15.3) |

BALB/c mice were infected by retro-orbital injection with 1×108 CFU of S. aureus Newman or its variants with insertional lesions in either sortase A (srtA) or any one of eighteen genes encoding sortase A-anchored surface proteins or the two coagulase genes, coa and vwb. Median survival time represents the time at which 50% of infected mice (n=10) exhibited lethal disease. Statistical significance was determined by the two-tailed logrank test. Data are representative of two independent experiments.

### Example 3

### Genetic Requirements for Staphylococcal Agglutination

S. aureus Newman mutants with defined genetic lesions (Base et al., 2004) were screened for defects in agglutination (FIG. 1A). Mutations that abrogated the secretion of only one of the two coagulases, Coa (Kaida et al., 1987) or vWbp (Bjerketorp et al., 2002), had little or no effect on agglutination (FIG. 1AB). In contrast, a mutant lacking both genes (coa/vwb) was severely impaired for agglutination, similar to a clfA variant (FIG. 1AB). A mutant lacking all three genes—coa, vwb, and clfA—was unable to agglutinate in plasma (FIG. 1AB). Mutants with insertional lesions in other known fibrinogen binding proteins, efb (Palma et al., 1996; Palma et al., 1998) and clfB (Ni Eidhin et al., 1998), did not cause large defects in agglutination (FIG. 1AB). The phenotypic agglutination defects of coa/vwb as well as clfA mutants could be restored by transformation of staphylococci with pcoa-vwb and pclfA, respectively, plasmids encoding wild-type alleles to the corresponding mutational lesions (FIG. 1C). Thus, unlike ClfA-mediated clumping of staphylococci via binding to fibrinogen (McDevitt et al., 1994), S. aureus agglutination appears to be a multi-factorial process involving coagulases, ClfA, as well as fibrinogen and prothrombin (Birch-Hirschfeld, 1934).

### Example 4

### Staphylococcal Agglutination in Septic Mice

To test whether staphylococcal agglutination occurred in mice with sepsis, the hearts of animals that had succumbed to S. aureus Newman challenge were examined for histopathology (FIG. 2). Deposits of large numbers of staphylococci, mostly without immune cell infiltrates, were identified in hematoxylin-eosin stained heart tissue twelve hours after infection (FIG. 2A-D). The appearance of these staphylococcal agglutinations is consistent with the general concept of thromboembolic deposition of S. aureus during sepsis (Hawiger et al., 1975) (FIG. 2A). Immuno-histochemical staining was used to detect specific agglutination factors (FIG. 2E). These experiments identified prothrombin and fibrinogen (fibrin) in the immediate vicinity of staphylococcal agglutinations (FIG. 2E). In agreement with the hypothesis that agglutination contributes to the pathogenesis of sepsis, fewer heart lesions were observed when mice were challenged with either clfA or coa/vwb variants (FIG. 3). Of note, heart tissues of animals necropsied twelve hours after intravenous challenge harbored considerable loads of staphylococci, irrespective of the challenge strain. Nevertheless, histopathology features of heart lesions associated with clfA or coa/vwb variants revealed immune cell infiltrates in the absence of staphylococcal agglutinations (FIG. 3B). A mutant lacking all three agglutination factors—clfA, coa and vwb—failed to generate either immune cell infiltrates or S. aureus agglutinations in heart tissues (FIG. 3B) and appeared avirulent in the mouse sepsis model (FIG. 3C).

### Example 5

### Clumping Factor A Tethers Staphylococci to Fibrin Cables

Staphylococcal agglutination requires coagulase catalyzed conversion of fibrinogen to fibrin as well as ClfA-mediated attachments. If so, ClfA may bind not only fibrinogen but also fibrin. This prediction was tested by measuring the binding of purified recombinant ClfA to either fibrinogen or fibrin immobilized in wells of polystyrene plates (FIG. 4A). Using non-linear regression analyses, we calculated a dissociation constant (Kd) of 395.2 nM (±51.82) for ClfA binding to fibrinogen, comparable to earlier affinity measurements (McDevitt et al., 1997). The Kd of ClfA binding to fibrin was calculated as 661.9 nM (±80.32), which is not significantly different from the affinity of ClfA for fibrinogen (FIG. 4A). To further investigate S. aureus Newman interactions with fibrin, staphylococci were examined by scanning electron microscopy (SEM), which revealed agglutinated wild-type bacteria enmeshed in fibrin cables (FIG. 4B). SEM analysis of the staphylococcal variants coa/vwb and coa/vwb/clfA identified bacteria without fibrin cables (FIG. 4B). The clfA mutant continued to convert fibrinogen to fibrin, however clfA variant staphylococci did not agglutinate with fibrin cables (FIG. 4B). Plasmids pcoa-vwb and pclfA complemented the phenotypes caused by mutations in the corresponding genes and restored staphylococcal agglutination to wild-type levels (FIG. 4B). These data are in agreement with our general hypothesis that Coa/vWbp-derived fibrin cables provide a tether for ClfA-mediated staphylococcal agglutination (FIG. 4B).

### Example 6

### Antibodies that Prevent Staphylococcal Agglutination and Sepsis

To further explore the contributions of Coa, vWbp and ClfA to staphylococcal agglutination, we raised rabbit antibodies against affinity purified recombinant proteins (Cheng et al., 2010; Stranger-Jones et al., 2006). Affinity purified rabbit antibodies specific for Coa, vWbp or ClfA inhibited S. aureus Newman agglutination in plasma (FIG. 4C). Passive transfer of ClfA-specific rabbit antibodies (85 μg purified antigen-specific IgG) into the peritoneal cavity of mice reduced the deposition of S. aureus Newman agglutinations in heart tissues of infected animals (FIG. 5A). Active immunization of mice with purified Coa and vWbp or ClfA raised specific IgG antibodies and reduced the frequency of heart lesions in animals challenged for twelve hours with wild-type S. aureus Newman (FIG. 5B). In particular, the abundance of staphylococcal agglutinations without immune cell infiltrates was reduced (FIG. 5B). Active immunization of mice with all three antigens—Coa, vWbp and ClfA—eliminated staphylococcal agglutination in heart tissues and caused the largest reduction of all types of pathological lesions (FIG. 5B). Similar to Coa- and vWbp-specific immunoglobulin (Cheng et al., 2010), passive transfer of ClfA-specific rabbit antibodies into the peritoneal cavity of mice increased the survival time in the sepsis model of infection (FIG. 4D). These data corroborate the concept that ClfA-specific antibodies can improve the outcome of S. aureus Newman sepsis (Hall et al., 2003).

### Example 7

### Direct Thrombin Inhibitors and Staphylococcal Sepsis

Univalent direct thrombin inhibitors, e.g. argatroban and dabigatran, inhibit the proteolytically active Coa-prothrombin complex (Hijikata-Okunomiya and Kataoka, 2003; Vanassche et al., 2010). The inventors examined whether these inhibitors also block the catalytic activity of vWbp-prothrombin. As a control, conversion of fibrinogen to fibrin by thrombin was monitored as an increase in sample absorbance at 450 nm. Compared to a mock control, this reaction was blocked with 200 ng argatroban (FIG. 6A). Treatment of fibrinogen with either Coa-prothrombin or vWbp-prothrombin led to fibrin conversion, whereas incubation with prothrombin alone did not (FIG. 6A). Incubation of both Coa-prothrombin or vWb-prothrombin with 200 ng argatroban blocked the conversion of fibrinogen to fibrin (FIG. 6A). Argatroban treatment also interfered with the agglutination of S. aureus Newman in plasma (FIG. 6B).

To evaluate the efficacy of direct thrombin inhibitors on the outcome of S. aureus Newman sepsis, mice received intraperitoneal injections with 10 mg/kg dabigatran-etexilate in 12 hour intervals. Dabigatran-etexilate is converted in mammalian tissues to its active form, dabigatran, which acts as a direct inhibitor of thrombin (Haul et al., 2002). To assess dabigatran activity, mouse blood samples were drawn by cardiac puncture and the dilute thrombin time was determined (FIG. 9). Following challenge of mice via blood stream injection of 1×108 CFU S. aureus Newman, mock treated animals died of sepsis within 60 hours post challenge (FIG. 6C). In contrast, dabigatran-etexilate treated animals survived up to 132 hours, albeit that all animals in this cohort eventually succumbed to the challenge (FIG. 6C). To determine whether direct thrombin inhibitors specifically block Coa and vWbp, mock or dabigatran-etexilate treated animals were challenged with the S. aureus coa/vwb mutant. In these experiments, dabigatran-etexilate treatment had no effect on survival or time-to-death (FIG. 6C). Mock or dabigatran-etexilate treated mice were also infected with lethal doses of S. aureus USA300 LAC, the current clone responsible for the epidemic of community-acquired MRSA infections in the United States (DeLeo et al., 2010). Dabigatran-etexilate treatment prolonged the survival of septic mice (FIG. 6D).

### Example 8

### Inhibiting Multiple Staphylococcal Factors Improves the Outcome of Sepsis

If clfA, coa and vwb act together to promote S. aureus Newman agglutination, dabigatran-etexilate treatment would be expected to improve the outcome of sepsis caused by clfA mutant staphylococci (FIG. 7A). Indeed, dabigatran-etexilate treatment increased the survival and time-to-death of mice with sepsis caused by clfA mutant S. aureus compared to a control strain harboring the complementing plasmid pclfA (FIG. 7A). Dabigatran-etexilate treatment further improved the disease outcome of animals challenged with clfA mutant staphylococci compared to a cohort of mock treated mice (FIG. 7A). Injection of clfA mutants carrying pclfA into the blood stream of mice resulted in reduced time-to-death compared to the wild-type parent, S. aureus Newman (FIG. 7A). Nevertheless, animals infected with the clfA (pclfA) variant also benefited from dabigatran-etexilate treatment (FIG. 7A).

To test whether combining dabigatran-etexilate and ClfA-specific antibodies can improve the outcome of staphylococcal sepsis, animals received both treatments followed by challenge with a lethal dose of S. aureus (FIG. 7B). As compared to mock-treated animals or mice receiving either dabigatran or ClfA-specific antibodies, the combination of dabigatran and ClfA-specific antibodies led to increased time-to-death and survival of staphylococcal sepsis (FIG. 7BC).

Whether the use of thrombin inhibitors and ClfA-specific antibodies could aid in the prevention of sepsis caused by clinical S. aureus isolates was evaluated. To test this, the community-acquired MRSA isolate MW2, which was isolated from a fatal case of septicemia (Baba et al., 2002), as well as the hospital-acquired MRSA isolate N315 (Kuroda et al., 2001) were used. S. aureus strains N315 and MW2 both agglutinated when suspended in EDTA-plasma (FIG. 8A). These reactions were inhibited by treatment with argatroban (FIG. 8A) or with ClfA-specific antibodies (FIG. 8B). Treatment of mice with both dabigatran and ClfA-specific antibodies led to increased time-to-death during sepsis caused by either S. aureus N315 or S. aureus MW2 (FIG. 8CD). In contrast, the use of either dabigatran or ClfA-specific antibodies alone did not prolong the survival of mice receiving a lethal challenge of S. aureus N315 or S. aureus MW2 (FIG. 8CD).

### Example 9

### Treatment with Dabigatran-Etexilate Reduces Staphylococcal Agglutination in Heart Tissues During Sepsis

The effect of dabigatran-extilate on heart tissue infected with S. aureus Newman was evaluated using histopathology. Dabigatran-extilate reduced agglutination of staphylococcus in this tissue (FIG. 10).

### REFERENCES

The following references, to the extent that they provide exemplary procedural or other details supplementary to those set forth herein, are specifically incorporated herein by reference.
U.S. Pat. No. 4,415,723
U.S. Pat. No. 4,458,066
U.S. Pat. No. 4,554,101
U.S. Pat. No. 4,816,567
U.S. Pat. No. 5,889,136
U.S. Pat. No. 6,692,739
U.S. Pat. No. 6,979,446
U.S. Pat. No. 7,045,131
U.S. Pat. No. 7,364,738
U.S. Ser. No. 61/508,430
U.S. Publn. 2011/0020323
U.S. Publn. 2006/0222651
Ausubel et al., Current Protocols in Molecular Biology, Greene Publ. Assoc. Inc. & John Wiley & Sons, Inc., MA, 1996.
Baba et al., Lancet., 359:1819-1827, 2002.
Baba et al. J. Bacteriol., 190:300-310, 2007.
Bae et al., Proc. Natl. Acad. Sci. USA, 101:12312-12317, 2004.
Birch-Hirschfeld, Klinische Woschenschrifi, 13:331, 1934.
Bjerketorp et al., Microbiology, 148:2037-2044, 2002.
Bosher and Labouesse, Nat. Cell. Biol., 2(2):E31-E36, 2000.
Brummelkamp et al., Cancer Cell, 2:243-247, 2002.
Brummelkamp et al., Science, 296(5567):550-553, 2002.
Cheng et al., PLoS Pathogens, 6:e1001036, 2010.
Cheng et al., Trends Microbiol. 19(5):225-232, 2011.
Clackson et al., Nature 352: 624-628, 1991.
DeDent et al., EMBO J., 27:2656-2668, 2008.
DeLeo et al., Lancet., 375:1557-1568, 2010.
Diep et al., Lancet., 367:731-739, 2006.
Doolittle, Blood Rev., 17:33-41, 2003.
Elbashir et al., Genes Dev., 5(2):188-200, 2001.
Fire et al., Nature, 391(6669):806-811, 1998.
Fowler et al., JAMA, 293:3012-3021, 2005.
Friedrich et al., Nature, 425:535-539, 2003.
Ganesh et al., PLoS Pathog., 4:e1000226, 2008.
Grishok et al., Science, 287:2494-2497, 2000.
Hair et al., Infect. Immun., 78:1717-1727, 2010.
Hall et al., Infect. Immun., 71:6864-6870, 2003.
Harlow and Lane, In: Antibodies: A Laboratory Manual, Cold Spring Harbor Laboratory, 1988.
Haul et al., J. Med. Chem., 45:1757-1766, 2002.
Hawiger et al., Biochemistry, 21:1407-1413, 1982.
Hawiger et al., Nature, 258:643-645, 1975.
Hijikata-Okunomiya and Kataoka, Thromb. Haemost., 1:2060-2061, 2003.
Josefsson et al., J. Infect. Dis., 184:1572-1580, 2001.
Kaida et al., J. Biochem., 102:1177-1186, 1987.
Ketting et al., Cell, 99(2):133-141, 1999.
Kim et al., Vaccine, 28:6382-6392, 2010.
Klevens et al., JAMA, 298:1763-1771, 2007.
Kohler and Milstein, Nature, 256:495-497, 1975.
Kolle and Otto, Z. Hygiene, 41:369-379, 1902.
Kuroda et al., Lancet., 357:1225-1240, 2001.
Kyte and Doolittle, J. Mol. Biol., 157(1):105-132, 1982.
Lin and Avery, Nature, 402:128-129, 1999.
Loeb, J. Med. Res., 10:407, 1903.
Lowy, New Engl. J. Med., 339:520-532, 1998.
Marks et al., J. Mol. Biol. 222: 581-597, 1991.
Mazmanian et al., Science, 285:760-763, 1999.
McDevitt et al., Eur. J. Biochem., 247:416-424, 1997.
McDevitt et al., Mol. Microbiol., 11:237-248, 1994.
Montgomery et al., Proc. Natl. Acad. Sci. USA, 95:15502-15507, 1998.
Moreillon et al., Infect. Immun., 63:4738-4743, 1995.
Palma et al., Infect. Immun., 64:5284-5289, 1996.
Palma et al., J. Biol. Chem., 273:13177-13181, 1998.
Ni Eidhin et al., Mol. Microbiol., 30:245-257, 1998.
Remington's Pharmaceutical Sciences, 15^{th} Ed., 1035-1038 and 1570-1580, 1990.
Sambrook et al., In: Molecular cloning, Cold Spring Harbor Laboratory Press, Cold Spring Harbor, N.Y., 2001.
Schneewind et al., EMBO J, 12:4803-4811, 1993.
Sharp and Zamore, Science, 287:2431-2433, 2000.
Sharp, Genes Dev., 13:139-141, 1999.

Stranger-Jones et al., Proc. Natl. Acad. Sci. USA, 103:16942-16947, 2006.
Strong et al., Biochemistry, 21:1414-1420, 1982.
Sui et al., Proc. Natl. Acad. Sci. USA, 99(8):5515-5520, 2002.
Tabara et al., Cell, 99(2):123-132, 1999.
Vanassche et al., J. Clin. Microbiol., 48:4248-4250, 2010.
Verme-Gibboney et al., Ann. Pharmacother., 37(7-8):970-975, 2003.
Weems Jr., et al., Antimicrob. Agents Chemother., 50:2751-2755, 2006.
Wincott et al., Nucleic Acids Res., 23(14):2677-2684, 1995.

1. A method for treating or preventing a Staphylococcus bacteria infection in a subject comprising administering to the subject effective amounts of a ClfA inhibitor and one or more thrombin inhibitors.

2. The method of claim 1, wherein the subject has been exposed to Staphylococcus bacteria or is at risk for exposure to Staphylococcus bacteria.

3. The method of claim 1, wherein the Staphylococcus bacteria is methicillin-resistant.

4. The method of claim 1, wherein the subject is determined to be infected with Staphylococcus bacteria.

5. The method of claim 4, wherein the subject is administered the ClfA inhibitor and one or more thrombin inhibitors within 12 hours of being determined to be infected with Staphylococcus bacteria.

6. The method of claim 1, wherein the thrombin inhibitor is argatroban, dabigatran, dabigatran-etexilate, melagatran, ximelagatran, efegatran, hirudin, bivalirudin, odiparcil or efegatran-sulfate.

7.-10. (canceled)

11. The method of claim 1, wherein the ClfA inhibitor comprises an antibody or antibody fragment that specifically binds ClfA.

12. The method of claim 11, wherein the ClfA inhibitor comprises chimeric, humanized, scFv, and/or bi-specific antibody.

13. The method of claim 12, wherein the ClfA inhibitor comprises a chimeric or humanized antibody.

14. The method of claim 1, wherein the ClfA inhibitor and the one or more thrombin inhibitors are administered within 30 minutes of each other.

15. The method of claim 14, wherein the ClfA inhibitor and the one or more thrombin inhibitors are administered at the same time to the subject.

16.-18. (canceled)

19. The method of claim 1, further comprising administering to the subject an antibiotic.

20.-21. (canceled)

22. The method of claim 1, wherein the subject is administered 0.01 μg to 10 mg of the one or more thrombin inhibitor.

23.-24. (canceled)

25. The method of claim 1, wherein the ClfA inhibitor blocks the interaction of ClfA with fibrin.

26. The method of claim 1, wherein the ClfA inhibitor blocks the interaction of ClfA with fibrinogen.

27. A composition comprising at least one thrombin inhibitor and a ClfA inhibitor.

28. The composition of claim 27, comprising a thrombin inhibitor selected from the group consisting of argatroban, dabigatran, dabigatran-etexilate, melagatran, ximelagatran, efegatran, hirudin, bivalirudin, odiparcil or efegatran-sulfate.

29. The composition of claim 28, comprising the thrombin inhibitor dabigatran-extexilate.

30. The composition of claim 29, wherein the ClfA inhibitor comprises an antibody or antibody fragment that specifically binds ClfA.

31.-32. (canceled)

33. A composition comprising a ClfA inhibitor, a Coa inhibitor and a vWbp inhibitor.

34. (canceled)
